(19) United States
(12) Patent Application Publication
(10) Pub. No.: US 2006/0264414 A1
(43) Pub. Date: Nov. 23, 2006

(54) Fluorous 2,2'-bipyridines and fluorinated biphasic sysems for ligand design

(75) Inventors: Stephen Carper, Las Vegas, NV (US); Byron Bennett, Ormond Beach, FL (US)

Correspondence Address: MARK A. LITMAN & ASSOCIATES, P.A.;York Business Center, Suite 205, 3209 W. 76th St., Edina, MN, 55435, US

(73) Assignees: The Board of Regents, University and Community College System of Nevada on behalf of the; University of Nevada - Las Vegas

(21) Appl. No.: 11/400,886

(22) Filed: Apr. 10, 2006

Related U.S. Application Data
(60) Provisional application No. 60/669,477, filed on Apr. 8, 2005.

(51) Int. Cl. A61K 31/555; C07F 15/00

(52) U.S. Cl. 514/184; 514/185; 546/2

(57) ABSTRACT

A series of 2,2′-bipyridines or 1,10-phenanthrolines featuring alkyl groups appended in the 4,4′ positions or 4,7-positions have been prepared. There is an insulating role of the methylene spacers as the electrochemical reduction potentials of these compounds that is almost identical to that of 2,2′-bipyridine. Calculations for (CH₂)ₙCF₃ derivatives (n=0-10) describes a limit for impact of CF₃ through 9-10 methylenes. From both physical and theoretical data (CH₂)₃(CF₂)ₓ₋₁CF₃ alkyl groups are inductively equivalent to hydrogen. Complexes of the present invention have the formula cis-LPtCl₂ where L is a 4,4′-substituted-2,2′-bipyridine or a 4,7-substituted-1,10-phenanthroline. The substituents may preferably be normal, branched and cyclic alkyl groups, alkyl groups with ether linkages, highly fluorinated alkyl group, highly fluorinated alkyl groups with ether linkages, hydroxyl terminated alkyl groups, hydroxyl-terminated alkyl groups with ether linkages and perfluorinated alkyl groups.

### CROSS-REFERENCE TO RELATED APPLICATIONS, IF ANY

This application claims the benefit under 35 U.S.C. §119 (e) of provisional application Ser. No. 60/669,477, filed 8 Apr. 2005.

### BACKGROUND OF THE INVENTION

1. Field of the Invention

The present invention relates to the synthesis of and use of classes of compounds in the treatment of disease, including cancer.

2. Background of the Art

Photodynamic therapy (PDT) has been used widely in the treatment of a variety of cancers, including breast metastases, gynecological tumors, cutaneous cancers, Karposi's sarcoma, and papillomatosis. In general, PDT has involved localization of a chromophoric dye molecule (e.g., porphyrins, chlorins, pheophorbides, and phthalocyanines) at a cancerous site, followed by optical excitation of the dye at relatively long wavelengths (e.g., λ=650 nm or higher), where the transparency of human tissue is significant.

Particularly, the chromophore-containing dye molecule in its natural or ground state is a singlet (i.e., ¹ππ) such that the two electrons in the highest occupied molecular orbital are paired. Upon optical excitation, the photoexcited ¹ππ* state of the dye molecule decays non-radiatively to a triplet (i.e., ³ππ*) state, which is lower in energy than the photoexcited singlet state of the dye molecule. In its triplet state, the dye molecule is then reacted with oxygen, which in its ground state is a triplet (i.e., ³O₂). By way of a triplet-triplet annihilation mechanism, the excited triplet state of the dye molecule and the ground state (triplet) oxygen react to produce the ground state dye molecule and oxygen in the singlet state (i.e., ¹O₂), which is non-selectively cytotoxic. In this manner, existing PDT methods attempt to control the chemical decomposition of carcinogenic cells through selective optical initiation at wavelengths exhibiting moderate tissue penetration.

In the PDT procedures, a therapeutically effective amount of a compound capable of forming a radical upon exposure to light by a unimolecular mechanism (e.g., in the absence of oxygen) is administered (e.g., by injection) to a patient so as to contact the cancer or microorganism selected for treatment. The compound is then irradiated at the site of action so as to induce radical formation. Strictly by way of example, the compounds can include metalloenediynes (i.e., transition metal complexes with metal chelating enediyne ligands) and/or transition metal complexes that bear at least one diazo functional group, such as, but not limited to, a terminal diazo group or as in a triazine (also generally referred to herein as “transition metal diazo compounds” or “transition metal diazo complexes”).

It is known that numerous transition metal complexes, particularly palladium and platinum complexes, have a chemotherapeutic activity, as described in U.S. Pat. No. 4,584,316. However, the complexes used at present have a therapeutic index (efficacy/toxicity ratio) which is still too low. Their excessive toxicity limits their use, notably on account of the risk of renal lesions. One way of reducing this major disadvantage is to “isolate” these complexes by incorporation into or association with a vector, permitting a slower diffusion of the active principal. The encapsulation into liposomes of cis-platinum (Freise, J., W. H. Mueller, P. Magerstedt, H. J. Schmoll (1982) Arch. Int. Pharmacodyn., 258, 180) and analogs thereof (Khokhar, A. R., S. Al-Baker, R. Perez-Soler (1988) Anticancer Drug Design 3, 177), reduces the efficacy of these agents, but improves the therapeutic index, prolongs their action, favorably modifies their biodistribution, and even promotes the induction of an antitumor activity against resistant tumors.

Cisplatin (cis-diamminedichloroplatinum, cis-Pt(NH.sub.3).sub.2 Cl.sub.2, molecular weight 300.05) has been used as a chemotherapeutic agent for many years since the discovery of its anti-tumor activity by B. Rosenberg et. al. (Nature, 1965, 205, 698; Nature, 1972, 222, 385).

Chemical & Engineering News (Oct. 23, 1995) reported that “Cisplatin was first synthesized in the 1800s, but its anticancer activity was not discovered until the 1960s. In 1979, it was approved by the Food and Drug Administration for clinical treatment of testicular and ovarian tumors and cancers of the head and neck. Cisplatin and an analog, carboplatin, are now among the most widely used anticancer drugs.”

The Physician's Desk Reference reports that cisplatin (the commercial name is Platinol®) can be used to treat testicular cancer, ovarian cancer, and bladder cancer. Rosenberg et al., U.S. Pat. No. 4,177,263, describes methods of treating cancer using cisplatin and cisplatin analogs. The compound was shown to be effective for treating leukemia and tumors induced in mice.

After so many years, cisplatin is still being widely used because of its efficacy. However, its critical drawback, the toxicity, is still a major concern. Many attempts have been made to either reduce its toxicity or increase its efficacy.

Predominantly, cisplatin binds onto deoxyguanosine of DNA. It also binds onto other deoxynucleosides or nucleosides. Because of the non-selectivity of cisplatin between cancer cells and normal cells, cisplatin has a lot of side effects. Besides, cisplatin is effective only to certain kinds of cancers. Therefore, reducing the toxicity of cisplatin and expanding its use in more cancers have been very important issues for all scientists involved in its research.

Many people have attempted to change the ligand on platinum to make new cisplatin analogs in order to reduce the toxicity or improve the efficacy. Examples are made by K. C. Tsou, et al.(J. Clin. Hemat. Oncol. 1977, 7, 322,), R. J. Speeder et al. (J Clin. Hemat. Oncol. 1977, 7, 210), A. Mathew et. al. (Chem. Comm. 1979, 222), D. Rose, et al. (Cancer Treatment Reviews, 1985, 12, 1), and D. Alberts et al. (Cancer Treatment Reviews, 1985, 12, 83).

U.S. Pat. Nos. 5,648,362 and 5,399,694 (Riess et al.) address perceived deficiencies in such cis-platinum compound delivery by using fluoroalkylated amphiphilic ligands derived from aromatic amines of the bipyridine (I) or phenanthroline (II) types, and form complexes with platinum, palladium and ruthenium. In their disclosed chemical formulae (I) and (II), R¹ and R² are independently a hydrogen atom, or a fluoroalkylated or hydrocarbon chain, provided at least one of R¹ and R² is a fluoroalkylated chain, and W represents a methylene, ester, ether, carbonyl or amide group. Fluoroalkylated ligands (I or II) and their complexes can be included in preparations comprising emulsions, dispersions, gels, or microemulsions, particularly in preparations for therapeutic use. In the earlier journal reports [e.g., Journal of Organic Chemistry (1992) vol. 57 page 3046ff and European Journal of Medicinal Chemistry (1993) vol. 28 pages 235-242] a clear deficiency in the scope of the technology is evident.

1) In general the goal of the authors was to utilize the fluorophilicity of the new ligands and corresponding complexes to deliver Pt and Pd to tissues in emulsions. To accomplish this task a significant fluorine “loading” had to be done to assure that desired physical properties are observed. Thus the authors mention 50% fluorine content in the ligand as a design criteria for the bipyridine and phenanthroline ligands. It is on this basis that the (U.S. Pat. No. 5,648,362) that the ligand group is defined with the fluoroalkyl substituents having two carbons or more (RF groups) [Column 4 lines 40-65]. The equivalent statement from claim 2 in U.S. Pat. No. 5,399,694 is quoted below. “wherein R_{F} is a branched, cyclic, or linear fluorinated alkyl radical of from 2 to 23 carbon atoms in which 50 to 100% of the hydrogen atoms have been replaced by fluorine atoms, and R_{F} comprises at least 4 fluorine atoms, and optionally can bear other substituents chosen from Cl and Br; . . . ” 2) The authors mention in the Journal of Organic Chemistry report that they were not successful at preparing the saturated alkyl chain ligands via the dianion of 4,4-dimethyl-2,2′-bipyridine (p 3048). It is specifically stated that “The preparation of 4,4′-bis[(F-Alkyl)alkyl]-2,2′bipyridines containing saturated hydrocarbon spacers between the bipyridine ring and the perfluoroalkyl tails (compound 6), avoiding the above problem of E/Z isomers, has been investigated. However, none of our experiments proved effective in the synthesis of such derivatives.”

Academic literature for related derivatives exists (1997-) supporting the observation of route functionality later presented herein. These related derivatives have three or four methylene “spacers” between the bipyridine ring and a fluorocarbon tail of 6-10 carbons in length. U.S. Pat. No. 6,875,886 (Frangioni) describes reagents and methods for diagnosis, detection and treatment of cancers (for example, prostate cancers). In particular, the invention provides methods to generate various functionalized (prostate-specific membrane antigen (PSMA) ligands, and their uses in diagnosis, detection, imaging, and treatment of prostate cancers, especially those overexpressing PSMA.

“Cytotoxic activity of new amphiphilic perfluoroalkylated bipyridine platinum and palladium complexes incorporated into liposomes.” Garelli, N.; Vierling, P.; Fischel, J. L.; Milano, G. Lab. Chim. Mol., Univ. Nice-Sophia Antipolis, Nice, Fr. European Journal of Medicinal Chemistry (1993), 28(3), 235-42. CODEN: EJMCA5 ISSN: 0223-5234. Journal CAN 119:130943 AN 1993:530943 CAPLUS, discloses the in vitro cytotoxic activity of 17 new perfluoroalkylated side-chain bipyridine Pt and Pd complexes, when incorporated into liposomes, was assessed against a human head and neck carcinoma cell line (CAL 27) sensitive to cisplatin. All the liposome-entrapped complexes inhibited cellular growth (IC50 values 5-154 □M and 2-84 □M for 5 h and 69 h of exposure, resp.). The most effective compds., 2 Pd complexes, had IC50 values of 5 and 10 □M for 5-h exposure) close to that of unencapsulated cisplatin (4 □M). The presence of perfluoroalkylated tails, previously shown to confer fluorophilicity and lipophobicity on the complexes, did not impair their cytotoxic activity.

Synthesis and characterization of amphiphilic platinum and palladium complexes linked to perfluoroalkylated side-chain disubstituted bipyridines. Garelli, Nathalie; Vierling, Pierre. Lab. Chim. Mol., Univ. Nice-Sophia Antipolis, Nice, Fr. Inorganica Chimica Acta (1992), 194(2), 247-53. CODEN: ICHAA3 ISSN: 0020-1693; CAN 117:183653; AN 1992:583653 CAPLUS, discloses the synthesis and characterization of cis-MLCl2 (M=Pd, Pt; L=4,4′-bis[(F-alkyl)alkyl]-2,2′-bipyridine, 4,4′-bis[(F-alkyl)alkyloxycarbonyl]-2,2′-bipyridine, 4,4′-bis[[2″-(F-alkyl)ethenyl]alkyloxycarbonyl]-2,2′-bipyridine) are described. These new amphiphilic perfluoroalkylated derivs., as potential antitumor agents, were designed to be incorporated into liposomes and, more particularly, into injectable fluorocarbon emulsions.

Riess, Jean G.; Vierling, Pierre; Garelli, Nathalie. Fluoroalkylated amphiphilic ligands, their metallic complexes and their uses. PCT Int. Appl. (1993), 55 pp. CODEN: PIXXD2 WO 9307123 A1 19930415 CAN 120:259964 AN 1994:259964 CAPLUS, discloses a wide range of properties and structural characterizations for a wide range of amphiphilic ligands, their metallic complexes and their uses, shown in Appendix I.

### SUMMARY OF THE INVENTION

A series of 2,2′-bipyridines and 1,10-phenanthrolines featuring alkyl groups, alkyl groups with ether linkages, highly fluorinated alkyl groups, high;y fluorinated alkyl groups with ether linkages, hydroxyl terminated alkyl groups, hydroxyl-terminate alkyl groups with ether linkages or perfluorinated alkyl groups [e.g., (CH₂)ₙ(CH₃); (CH₂)ₙ—O—(CH₂)CH₃; CH₂(CH₂)ₙ(CF₂)ₓ₋₁CF₃ (CClF)ₙ(CF₂)ₓ₋₁CF₃; (CH₂)ₙ(CH₂OH) wherein n=0-5, m=0-3; and x=1-6], such as 1, (1); 6, (2); 8, (3); 10 (4)] appended in the 4,4′ positions of 2,2′-bipyridines have been prepared for the 4- and 7-positions of 4,7-disubstituted-1,10-phenanthroline. Ab initio calculations of vertical ionization potentials (VIPs) for 1-4 confirm the insulating role of the methylene spacers as the electrochemical reduction potentials of 1-4 are almost identical to that of 2,2′-bipyridine. Calculations for (CH₂)ₙCF₃ derivatives (n=0-10) describes a limit for impact of CF₃ through 9-10 methylenes. From both physical and theoretical data (CH₂)₃(CF₂)ₓ₋₁CF₃ alkyl groups are inductively equivalent to hydrogen. Pt(II) complexes of the present invention have the formula cis-LPtCl₂ where L is a 4,4′-substituted-2,2′-bipyridine or a 4,7-substituted-1,10-phenanthroline. The substituents may preferably be —CH₃ or —CH₂CH₂CH₂CF₃.

### BRIEF DESCRIPTION OF THE FIGURE

FIG. 1 shows a graphic representation of Calculated Vertical Ionization Potentials for 2,2′-bpy with (CH₂)ₙCF₃ Substituents in the 4,4′-Position. The Symbol ♦ corresponds to HF-3-21G* values and symbol ♦ corresponds to B3LYP/6-31+G* values. Lines were inserted at the calculated values for species with CH₃ substituents at the 4,4′-position (HF: −8.53 eV, B3LYP: −8.13 eV).

### DETAILED DESCRIPTION OF THE INVENTION

Since the advent of Fluorous Biphasic System (FBS) catalytic schemes,^{i ii} rapid growth of a new class of ligands that incorporate long aliphatic fluorocarbon chains has taken place.ⁱⁱⁱ Ligand design for use in FBS protocol typically involves maximizing the number of fluorine atoms incorporated to ensure complete immobilization of the new ligand in the fluorous recovery solvent.^{iv v} The electronic impact (inductive electron-withdrawing effect) of the appended fluorocarbon substituent is not always desirable. Thus successful design and implementation of a FBS transition metal catalyst requires some means to insulate of the perfluoroalkyl chains from the catalytic center while maintaining the appropriate fluorine loading necessary for desirable solubility properties.

The long-range electronic impact of perfluoroalkyl groups through methylene spacers has been investigated for trialkylphosphines. There is an indication in the art (J. A. Gladysz, “Are Teflon “Ponytails” the Coming Fashion for Catalysts?”, Science, 1994, 266, 55. J. A. Gladysz and Dennis P. Curran, Tetrahedron, 2002, 58(20), 3823-3825. The complete issue is dedicated to current works with fluorocarbon-modified materials (p 3827-4131). Istvan T. Horvath, Acc. Chem. Res, 1998, 31, 641. M. Cavazzini, F. Montanari, G. Pozzi, and S. Quici, Journal of Fluorine Chemistry, 1999, 94, 183. Richard H. Fish, Chem. Eur. J., 1999, 5(6), 1677) that a minimum of 8-10 aliphatic methylene “spacers” are required to completely insulate a perfluoroalkyl group from a P(III) center. From a practical standpoint this ideal situation may not always be synthetically feasible or useful for FBS applications. Thus FBS ligand designs focused on generating molecules with significant fluorophilicity will by necessity feature a reduced number of insulating methylenes.

In addition to phosphines, 2,2′-bipyridines and 1,10-phenanthrolines serve an important role as transition metal catalyst supports and therefore embody an important target for FBS design. Several groups have constructed 2,2′-bipyridines with fluorous substituents at the 4,4′ positions and applied these to FBS applications. Examples include ruthenium and copper catalyzed oxidation reactions, Pd(II)/Pt(II) drug delivery to human tissues, and polymerization catalysis.(Jianhui Xia, Terri Johnson, Scott G. Gaynor, Krzysztof Matydaszewski and Joseph Desimone, Macromolecules, 1999, 32, 4802.) Our disclosure is interested in the application of a fluorous biphase as a novel reaction medium for photo-reduction processes facilitated by (bpy)Re(I) complexes, and this note provides detail of the preparation and characterization of the 4,4′-substituted-2,2′-bipyridines 1-4 shown in Scheme 1. Specifically the inductive effect of the 4,4′-substituents is evaluated by comparison to alkyl analogues in both experiment and in theory. Our conclusions describe a practical design stipulation for alkyl insulation of perfluoroalkyl substituents on the 2,2′-bipyridine moiety that can be extended to other synthetic targets.

This disclosure encompasses inventions including the syntheses of a group of new cisplatin analogs, and the use of these cisplatin analogs to treat cancer. Generically, the present technology includes a cis-platin complex and a method of treating a patient having cancerous cells affecting tissue comprising providing the cis-LPtCl₂ complex wherein L is a 4,4′-substituted-2,2′-bipyridine or a 4,7-substituted-1,10-phenanthroline compound comprising 2,2′-bipyridines and 1,10-phenanthrolines having highly fluorinated alkyl groups, alkyl ether groups, or terminal alcohol groups appended in the 4,4′ positions of the 2,2′-bipyridine to the affected tissue or 1,10-phenanthroline in the 4,7 positions. This is preferably done without encapsulation or carriage of the complex in a lipid or liposome, but effected in a suspended or dissolved format without fatty carriers. The concentration of the complexes administered locally may be effective in picogram quantities at the site (e.g., at least 1.0 picograms per square mm of cancerous cell environment, the environment being the entire volume region where cells have been identified and not the volume of cancerous cells themselves. Higher concentrations and amounts of the complex are likely to be administered, especially with general administration, with nanogram concentrations (e.g., 1.0 nanograms per 1.0 mm cancerous cell environment) and even milligram concentrations (e.g., 1.0 milligram per 1.0 mm cancerous cell environment). This amount is greatly increased over time (e.g., these amounts may be delivered over 10 seconds, thirty seconds, minutes, hours or days), as the treatments are not single events but either episodic or continuous provision techniques. The providing of the complex to the affected tissue may be performed by at least one of general administration (e.g., oral, intravenous, topical, transdermal, etc.) of the complex and affected tissue targeted administration of the complex (e.g., diffusion from a catheter, implantation, perfusion through a cather, injection, and the like). The term highly fluorinated alkyl group refers to a group that approximates the electronic withdrawing effects of a perfluorinated group, such as a perfluorinated group in which no more than a single fluorine atom in two adjacent carbon atoms in the alkyl chain have been replaced by chlorine or other similar moiety. The preferred highly fluorinated groups comprise perfluorinated alkyl groups. The complex may have L comprise a 4,4′-substituted 2,2′-bipyridine or a 4,7-substituted-1,10-phenanthroline. The complex may have the 4 and 4′ substituents or the 4,7 substituents symmetrical or asymmetrical with respect to each other. The perfluorinated group may be, by way of non-limiting examples selected from [(CH₂)₃(CF₂)ₓ₋₁CF₃ wherein x=1-10, and may be linear, branched or cyclic perfluorinated, and may have linking groups such as ether, thioether, and alkyl groups within the chain. The preferred compounds to date are where the perfluorinated alkyl group is selected from (CH₂)CF₃; (CH₂)₂CF₃; (CH₂)₃CF₃; and (CH₂)₄CF_{3,} such as 4,4′-bis(4,4,4-trifluorobutyl)-2,2′-bipyridine. Alkyl substituents with terminal perfluorinated groups are also useful, as exemplified immediately below.

The complexes of the present invention have the formula cis-LPtCl₂ where L is a 4,4′-substituted-2,2′-bipyridine or a 4,7-substituted-1,10-phenanthroline. The substituents may be —CH₃ or —CH₂CH₂CH₂CF₃. The starting materials for the derivatives are shown below as examples of the nomenclature, with what is ordinarily identified as methyl groups in the 4,4′-positions and the 4,7-positions actually indicating only the point of attachment of the R1 and R2 groups defined herein. In structure 2, below, the single bond shown on the 4-position is, in fact, a methyl group. That compound exhibits asymmetry, and may be an effective way of reducing the extreme activity associated with cis-platin compounds. That is, compounds may be provided with only a single highly fluorinated group-containing substituent (on either the 4 or 4′-position or the 4- and 7-position), while the other position (the 4′- or 4-position and the 7- or 4-position, respectively) may have an alkyl group (or even a hydrogen) that reduces the activity of the cis-platin to a more controlled level of cell toxicity.

The use of different terminal groups on the susbtituent alkyl groups in the 4,4′- and 4,7-positions has also been contemplated as only hydroxyl groups (OH) or cyano groups (—CN), as alternatives to one or both highly-flurorinated group containing substituents.

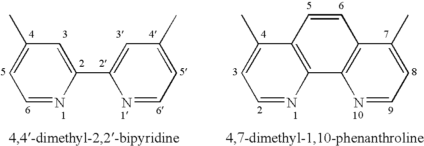

This class of cisplatin analogs maintains the original active sites of cisplatin (i.e., two Pt—Cl bonds in cis position). In addition, there are polar substituents at the terminus of each appended alkyl chain to assist in binding to the major and minor grove of DNA i.e., surrounding sugar/phosphate regions. Specific examples of making these complexes and their characterization are shown below. The complex of the present invention may be used to treat cancer tissue. Data is supplied describing potential utility in treatment of breast cancer. In particular in-vitro experiments compare breast cancer cell survival trials for complex I and cis-Platin.

These Cisplatin analogues will bind DNA with secondary intermolecular interactions (Hydrogen Bonding or electrostatic effects). Replacement of the amine ligands (NH₃) with ftinctionalized imine ligands (2,2′-bipyridine and 1,10-phenanthroline) affords the opportunity to retain the Pt binding site (for Guanine and Cytosine) and implement new polar groups at a remote position for interaction with the double helix groove wall (polar sugar and phosphate residues).

The scheme below exhibits the derivatives prepared to date. 1,2,and 3 have been characterized by NMR spectroscopy with satisfactory elemental analysis obtained for 1 ,2. The preparation of all the ligands (A, B, C) and complexes are novel. 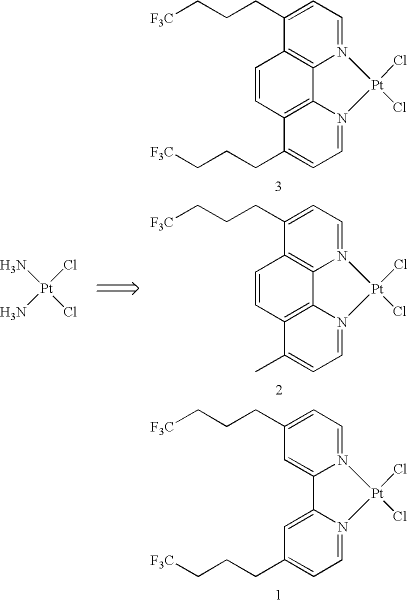
As noted elsewhere, all of the compounds described in the practice of this technology, including normal, branched and cyclic alkyl groups, alkyl groups with ether linkages, highly fluorinated alkyl group, highly fluorinated alkyl groups with ether linkages, hydroxyl terminated alkyl groups, hydroxyl-terminate alkyl groups with ether linkages and perfluorinated alkyl groups, may be synthesized by the selection of the appropriate reagent. Providing multiple and otherwise identical reaction paths would be superfluous for all of the alternatives.

Survival studies (Clonogenic Assay) of Breast Cancer Cells (DC4 and DB-46) treated with solutions of 1 show increased cell death when compared to identical experiments with Cisplatin. Plots are appended to this document that provide viable cells vs. concentration of Pt agent. Comparison of the 10 micromolar data demonstrates the enhanced reactivity of 1: the cisplatin treated samples have approximately 10% of the colonies surviving whereas samples treated with 1 have only 1% of the colonies surviving.

Flow cytometry results indicate the mechanism of cell death is not the same for samples treated with Cisplatin and 1.(Flow Cytometry Data is not appended to this report) Thus the potential for treatment of Cisplatin resistant cell lines with 1 is a reality. Given the structural similarity with 1, complexes 2 and 3 should also function by pathways similar to 1.

The novel Pt complexes are the result of novel ligand coordination to Pt(II). Thus the outline of ligand construction is given below followed by complex synthesis from the ligands.

Ligand Synthesis:

Essentially deprotonation of 4,4′-dimethyl-2,2′-bipyridine followed treatment with primary alkyl iodide (CF₃CH₂CH₂I) affords the ligands in modest to low yield. 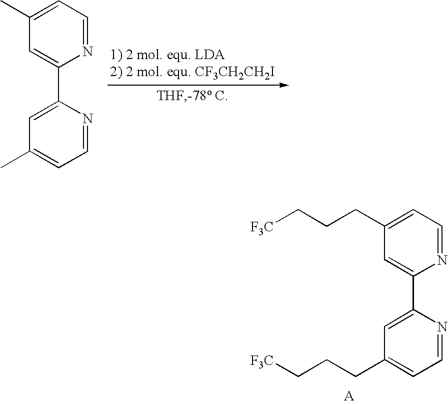
In the preparation of any compound of the invention, whether symmetrical or asymmetrical, the synthesis is performed by selection of the appropriate reagent. Where asymmetry is sought or results, this may be done by use of stoichiometric combinations of reagents, such as reagents having 4-substituents of R1 groups and 4′-substituents of R2 groups, where the R1 and R2 groups are to be the various and asymmetrical arms off the 2,2′-bipyridine or 1,10-phenanthroline nucleus. The product may be purified into the products by standard laboratory or commercial techniques. If the reaction rates are not identically equivalent, this will result in only a shift in the distribution, not in the absence of the asymmetrical product. A wide range of R1 and R2 groups (to be attached to the 4 and 4′ positions of the 2,2′-bipyridine, or the 4 and 7 positions of the 1,10-phenanthroline) may be used within the broad descriptions provided herein. Non-limiting examples of the R1 and R2 groups can include —(CF₂)ₙCF₃; —(CF₂)₂CF₃; —(CF₂)₃CF₃; (CF₂)₄CF₃ and analogs thereof; from —(CFCl)ₙCF₃; —(CF₂)₂CFClCF₃; —CFCl(CH₂)₃CF₃; and —C(CF₃)₃; tert-C CFCl(CF₃)₂ and —(CF₂)O(CH₂)CF₃; (CF₂)O(CF₂)₂CF₃; (CF₂)₃FClCF₃; and (CH₂)₄CF_{3,,} from (CH₂)ₙCF₃; (CH₂)₂CF₃; (CH₂)₃CF₃; and the like, wherein n is an integer of from 1 to 8, preferably from 1 to 6 or 1 to 4.

### Preparation of 4,4′-bis(4,4,4-trifluorobutyl)-2,2′-bipyridine, A

A three-neck 250 mL round bottomed flask was charged with 2.475 g [24.46 mmol] of diisopropylamine dissolved in 100 mL of THF. The ambient atmosphere in the flask was replaced with nitrogen and was cooled to −78° C. Addition of 8.78 mL of n-BuLi in hexanes (2.541M) [21.52 mmol] was completed over a 5-minute period via syringe. After 20 minutes the reaction mixture was warmed to 0° C., stirred for 30 minutes, then cooled to −78° C. 1.992 g [10.81 mmol] of 4,4′-dimethyl-2,2′-bipyridine, dissolved in 20 mL of THF, was added via syringe and stirring was continued for 3 hours after which 4.863 g [22.11 mmol] of 3,3,3-trifluoro-1-iodopropane dissolved in 50 mL of THF was added via syringe. After an hour the reaction mixture was allowed to warm slowly to ambient temperature overnight. The reaction was quenched with 150 mL of brine; the residue was extracted into Et₂O and dried over Na₂SO₄. Evaporation afforded crude product. After recrystallization from MeOH, 2.15 g [5.71 mmol] was isolated as a white solid (52.8% yield).

### Characterization of 4,4′-bis(4,4,4-trifluorobutyl)-2,2′-bipyridine, A

Elemental Analysis: Calcd for C₁₈H₁₈F₆N₂: C, 57.45; H, 4.82; N, 7.44. Found: C, 57.73; H, 4.74; N, 7.53. (Desert Analytics: sample IB #96)

NMR: ¹H (400 MHz, 298K, CDCl₃): δ 8.62 (d, ³J_{HH}=5.0 Hz 2H, C—H 6,6′) 8.28 (d, ⁴J_{HH}=1.0 Hz 2H, C—H 3,3′) 7.17 (d,d ³J_{HH}=5.0 Hz, ⁴J_{HH}=1.0 Hz 2H, C—H 5,5′) 2.81 (t, ³J_{HH}=7.7 Hz, 4H, CF₃CH₂CH₂CH₂—Ar) 2.14 (m,4H, CF₃CH₂CH₂CH₂—Ar) 2.01 (m,4H, CF₃CH₂CH₂CH₂—Ar)

¹³C{¹H}(100 MHz, 298K, CDCl₃): δ 156.45 (2C, 2/2′) 150.87 (2C, 4/4′) 149.50 (2C, 6/6′) 126.9(q, ¹J_{FC}=276 Hz, 2C CF₃CH₂CH₂CH₂—Ar) 123.97 (2C, 3/3′) 121.33 (2C, 5/5′) 34.35 (2C CF₃CH₂CH₂CH₂—Ar) 33.30 (q, ²J_{FC}=29 Hz, 2C CF₃CH₂CH₂CH₂—Ar) 22.73 (2C CF₃CH₂CH₂CH₂—Ar)

¹⁹F (376 MHz, 298K, CDCl₃): δ−71.17 (t, 6F, ³J_{HF}=10 Hz)

MS (EI: 70 eV, m/z) 376.35 (M⁺, 4%), 357.25 (M⁺-F, 1%), 307.35 (M⁺-CF₃, 6%), 293.25 (M⁺-CH₂CF₃, 13%), 280.30 (M⁺-CH₂CH₂CF₃, 100%), 265.30 (M⁺-CH₂CH₂CH₂CF₃, 2%),

UV-Vis (CH₂Cl₂, 25° C.) λ, nm (c: cm⁻¹,M⁻¹) 282.2 (18400), 249.2 (12500), 241.9 (12700).

IR (microcrystalline powder, cm⁻¹) v 3057 (w), 3027 (m), 3007 (m), 2958 (s), 2878 (s), 1931 (m), 1599 (s), 1557 (s), 1464 (s), 1397 (s), 1268 (s), 1142 (s), 1070 (m), 1019 (m), 975 (m), 911 (m), 839 (s), 816 (w), 784 (w), 764 (w).

Preparation of Ligand B:

The 4,7-substituted-1,10-phenanthroline derivatives were prepared in similar fashion. Treatment of 4,7-dimethyl-1,10-phenanthroline with 1.1 equivalent of LDA and subsequent addition of primary alkyl iodide affords B in modest to low yield.

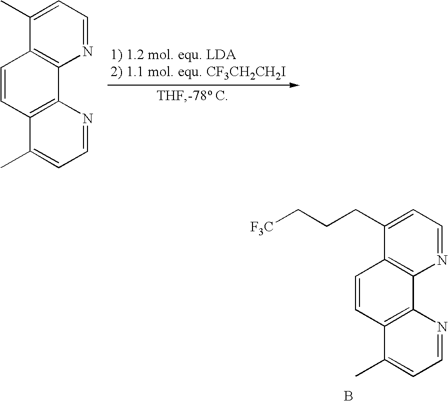

Characterization of Ligand B:(MRKI024)

NMR: ¹H (400 MHz, 295K, DMSO-d₆): δ 8.95 (m, 2H, C—H 2/9), 8.18 (m, 2H, C—H 5/6), 7.61 (m, 2H, C—H 3/8), 3.26 (t, ³J_{HH}=8 Hz, 2H, CF₃CH₂CH₂CH₂—Ar), 2.78 (s, 3H, Ar—CH₃), 2.42 (m, 2H, CF₃CH₂CH₂CH₂—Ar) 1.94 (m, 2H, CF₃CH₂CH₂CH₂—Ar).

Preparation of Ligand C:

Treatment of 4,7-dimethyl-1,10-phenanthroline with 2.1 equivalent of LDA and subsequent addition of primary alkyl iodide affords C in modest to low yield.

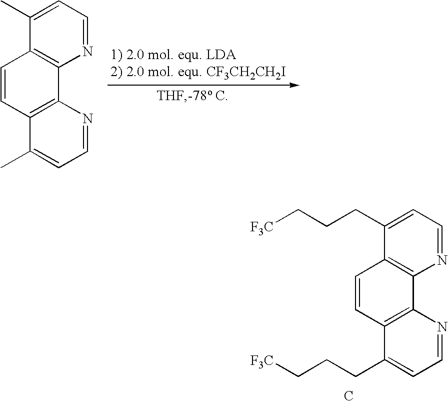

Characterization of Ligand C:

NMR: ¹H (400 MHz, 295K, DMSO-d₆): δ 8.99 (d, ³J_{HH}=4 Hz, 2H, C—H 2/9), 8.21 (s, 2H, C—H 5/6), 7.63 (d, ³J_{HH}=4 Hz, 2H, C—H 3/8), 3.28 (t, ³J_{HH}=8 Hz, 4H, CF₃CH₂CH₂CH₂—Ar), 2.43 (m, 4H, CF₃CH₂CH₂CH₂—Ar) 1.94 (m, 4H, CF₃CH₂CH₂CH₂—Ar).

Complex Synthesis:

Synthesis of the Pt(II) complexes is best accomplished by reaction of the ligands with 1,5-cyclooctadieneplatinumdichloride (COD)PtCl₂ in acetonitrile at reflux. The conversion is nearly quantitative (>90%) yielding yellow powders with low solubility in common organic solvents.

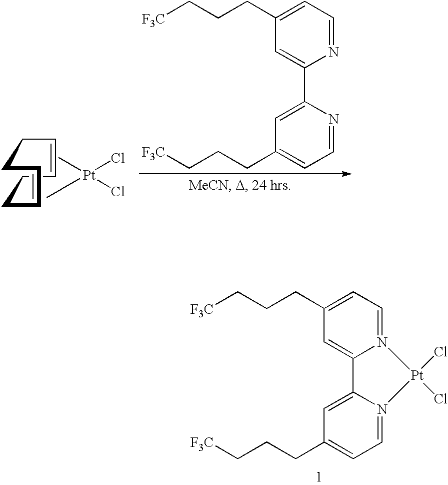

### Preparation of [4,4′-Bis(4,4,4-trifluorobutyl)-2,2′-bipyridine]PtCl₂, 1

A 100 mL round bottomed flask was charged with a stir bar, 35 mg (1,5-COD-²η-)PtCl₂ [0.0940 mmole], 39 mg 4,4′(Bis(4,4,4-trifluorobutyl)-2,2′-bipyridine [0.1036 mmole], and 15 mL of MeCN. The mixture was brought to reflux for approximately 24 hrs. Upon cooling the solvent was evaporated and residual COD removed via oil pump vac. The residue was then dissolved in MeCN and product precipitated with Et₂O. The yellow solid was recovered in 61% yield, 37 mg [0.0467 mmole].Elemental Analysis: Calculated for C₁₈H₁₈Cl₂F₆N₂Pt: C, 33.66%; H, 2.82%; N, 4.36%. Found: C, 33.81%; H, 2.80%; N, 4.25%. (Desert Analytics sample: BBI060)

¹H NMR (400 MHz, 295K, acetone-d₆) δ 9.513 (d, ³J_{HH}=6.0 Hz, 2H, —CH 6/6′), 8.467(s, 2H, —CH3/3′), 7.712(d, ³J_{HH}=6.0 Hz, 2H, —CH 5/5′), 2.999(t, ³J_{HH}=8.8 Hz, 4H, —CH₂CH₂CH₂CF₃), 2.379(m, 4H, —CH₂CH₂CH₂CF₃), 2.100(m, 4H, —CH₂CH₂CH₂CF₃);

¹³C{¹H} NMR (295 K, acetone-d₆) δ: 157.874 (CC, 2/2′,2C), 155.944 (CCH₂—, 4/4′, 2C), 149.036 (CH, CH 6/6′, 2C), 128.001 (CH, 3/3′, 2C), 125.030 (5/5′,2C), 128.397 (q, ¹J_{FC}=275.5 Hz, CF₃, 2C), 35.005 (—CH₂CH₂CH₂CF₃,2C) 33.45 (q, ²J_{FC}=28.5 Hz, —CH₂CH₂CH₂CF₃, 2C), 22.700 (q, ³J_{FC}=3.1 Hz, —CH₂CH₂CH₂CF₃, 2C)

¹⁹F NMR: (295 K, acetone-d₆) δ: −66.5 (t, ³J_{HF}=11.2 Hz, —CF₃, 6F) Reaction of ligands B and C with (COD)PtCl₂ affords the Pt complexes 2 and 3 under conditions analogous to those used for generating the 2,2′-bipyridine derivative affording yellow solids in nearly quantitative yield.

### Preparation of [4-CH₃,7-CF₃(CH₂)₃-1,10-phenanthroline]PtCl₂, 2 (BAII005)

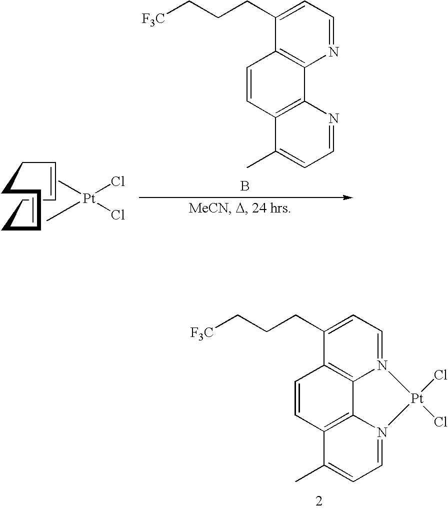

Characterization of Complex 2:

Elemental Analysis: Calculated for C₁₇H₁₅Cl₂F₃N₂Pt: C, 35.80%; H, 2.65%; N, 4.91%. Found: C, 33.51%; H, 2.71%; N, 4.83%.

NMR: ¹H (400 MHz, 295K, DMSO-d₆): δ 9.53 (m,2H, C—H 2/9), 8.38 (m, 2H, C—H 5/6), 8.01 (m, 2H, C—H 3/8), 3.26 (t, 2H, CF₃CH₂CH₂CH₂—Ar), 2.86 (s, 3H, Ar—CH₃), 2.49 (m, 2H, CF₃CH₂CH₂CH₂—Ar) 1.98 (m, 2H, CF₃CH₂CH₂CH₂—Ar).

### Preparation of [4,7-Bis(4,4,4-trifluorobutyl)-1,10-phenanthrolinelPtCl₂, 3

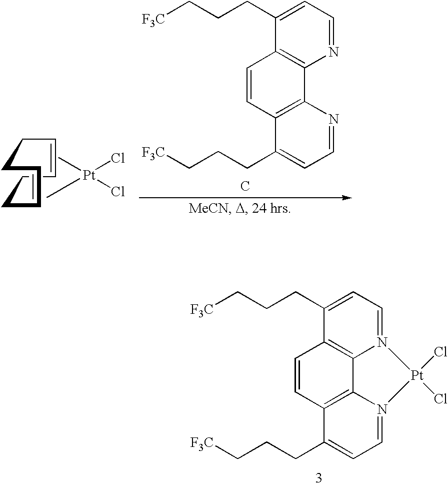

Characterization of Complex 3:

NMR: ¹H (400 MHz, 295K, DMSO-d₆): δ 9.54 (d, ³J_{HH}=8 Hz, 2H, C—H 2/9), 8.40 (s, 2H, C—H 5/6), 8.02 (d, ³J_{HH}=8 Hz, 2H, C—H 3/8), 3.28 (t, ³J_{HH}=8 Hz, 4H, CF₃CH₂CH₂CH₂—Ar), 2.47 (m, 4H, CF₃CH₂CH₂CH₂—Ar) 1.98 (m, 4H, CF₃CH₂CH₂CH₂—Ar).

As shown in Scheme 1 the preparation of 2,2′-bipyridines 1-4 features in-situ generation of the 4,4′-dilithioanion of 4,4′-dimethyl-2,2′-bipyridine followed by nucleophilic substitution of iodide from the appropriate fluorous primary alkyl iodide. Previous work utilizing this route reported modest yields. Xia et al. (Jianhui Xia, Terri Johnson, Scott G. Gaynor, Krzysztof Matyjaszewski and Joseph Desimone, Macromolecules, 1999, 32, 4802.} reported a 50% yield for ligand 2. A closely related molecule 4,4′-di-[(CH₂)₄(CF₂)₇CF₃]-2,2′-bipyridine has also been reported via the same route in 40% yield. We observe similar yields averaging between 30 and 40% for 1-4. To the best of our knowledge this is the first reported preparation of 1, 3 and 4.

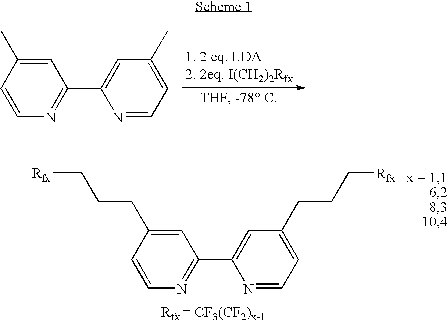

1-4 were characterized by physical and spectroscopic methods. Physical characterization included determination of melting points, fluorous partition data, and electrochemical reduction potentials in addition to elemental analysis and mass spectrometry. NMR (¹H, ¹³C{¹H}, ¹⁹F), IR and UV-Vis absorption spectra were also obtained for 1-4.

The white waxy solids, 1-4, each exhibit a simple melting endotherm as determined by differential scanning calorimetry (DSC): 1, 79.36° C.; 2, 113.14° C.; 3, 134.31° C.; 4, 156.95° C. The regular increase in the melting endotherm temperature in 1 to 4 reflects the gradual lengthening of the perfluorocarbon section or increasing value of “x” in the 4,4′ substituent —(CH₂)₃(CF₂)ₓ₋₁CF₃ (1, x=1; 2, x=6; 3, x=8; 4, x=10). No evidence for liquid-crystalline behavior was observed for these materials in the DSC traces.^{vi}

Fluorous partition coefficients (FPCs) were obtained for 1-4 for a 1:1 v/v biphasic mixture of “fluorous” solvent perfluoromethylcyclohexane, CF₃C₆F₁₁ and toluene. The phase content was determined using a GLC method and partition coefficients are shown in Table 1. Like 1 the data for the commercially available dimethyl and di-n-nonyl substituted derivatives show no compound in the CF₃C₆F₁₁ layer.

TABLE 1
Fluorous Partition Coefficients (FPCs) for 4,4′-substituted-2,2′-bipyridines

| 4,4′-Substituent | | % F | FPC | fᵢ^{a} |
|---|---|---|---|---|
| —(CH₂)₃R_{f1} | 1 | 30.3 | 0.00^{b} | — |
| —(CH₂)₃R_{f6} | 2 | 56.4 | 0.15 | −1.9 |
| —(CH₂)₄R_{f8} | | 58.5 | 0.67^{c} | −0.40^{c} |
| —(CH₂)₃R_{f8} | 3 | 60.0 | 0.84 | −0.17 |
| —(CH₂)₃R_{f10} | 4 | 62.5 | 11 | +2.4 |

FPC = C_{bpy}(CF₃C₆F₁₁)/C_{bpy}(toluene)
^{a}fᵢ = fluorophilicity = ln[FPC]
^{b}analyte not detected in the CF₃C₆F₁₁ layer
^{c}ref. 13 (Jianhui Xia, Terri Johnson, Scott G. Gaynor, Krzysztof Matyjaszewski and Joseph Desimone, Macromolecules, 1999, 32, 4802.)

The partition data in Table 1 clearly follow previous trends and “rules” observed for fluorocarbon modified organics as fluorine loadings >60% are required to impart significant fluorophilic character (FPC values>1). The FPCs determined for 3 and reported for the butyl spacer analogue⁸ reflect the decrease in fluorine loading resulting from addition of a single methylene unit to each tail. As expected the use of additional methylene spacers negatively impacts the overall fluorophilicity of the modified bipyridyl moiety. As a group 1-4 do not exhibit the extreme partition behavior required for classification as “heavy” fluorous materials typically utilized in classic FBS liquid-liquid separation schemes(FPCs>20, i.e. >95% resident in the CF₃C₆F₁₁ phase). However the ligand ensemble presented here will have significant applicability in “light” fluorous protocols.

The reduction potentials for 2,2′-bipyridine derivatives 1-3 and several reference compounds were determined by cyclic voltammetry at a 10 μm diameter Pt ultramicroelectrode (Table 2). THF was found suitable for this study as 1-3 have sufficient solubility and the electrochemical potential window (+1.2 V to −3 V vs. SCE) extends to the potential range of interest. The reductions of 4,4′-dimethyl-2,2′-bipyridine and 2,2′-bipyridine are known to occur at −2.68 V and −2.60 V respectively in DMF. The 80 mV difference observed in DMF is in good agreement with the 88 mV difference determined by our group in THF. The non-nemstian peak separations observed may be indicative of slow diffusion processes which have been suggested for fluorocarbon modified metallocene derivatives exhibiting similar separations.

TABLE 2
Cyclicvoltammetry for 4,4′-substituted-2,2′-bipyridines

| 4,4′-Substituient | E_{1/2} (V)^{a} | ΔEₚ (mV)^{b} |
|---|---|---|
| —(CH₂)₃R_{f1} (1) | −2.404 | 239 |
| —(CH₂)₃R_{f8} (3) | −2.405 | 288 |
| —H | −2.405 | 260 |
| —(CH₂)₃R_{f6} (2) | −2.407 | 291 |
| —CH₃ | −2.493 | 253 |
| —(CH₂)₈CH₃ | −2.501 | 299 |

^{a}Observed potentials are quoted relative to SCE using the ferrocene/ferrocenium couple as a reference[+0.310 V vs. SCE(aq) MeCN (0.2 M LiClO₄)] (see - Bard and Faulkner “Electrochemical Methods”, Wiley, 2^{nd} Edition, appendix)
^{b}Scan rate = 10 V/sec 01.M TBAPF₆

In order to provide an assessment of physical characteristics associated with the ligand class, we adopted a strategy previously employed for fluorous phosphine ligands. In this work, ionization energies for fluorous phosphine ligands were computed and compared with respect to incremental increases in the number of methylene spacers in the substituents. Calculations of VIPs were computed at the B3LYP/6-31G* level of theory, in which all neutral ligands were structurally optimized. A single point calculation at B3LYP/6-311+G* was carried out on the previously optimized structure. VIP values were then compared to the increase in the number of methylene spacers. Trends in the computationally determined VIP values demonstrated that the chains approach an asymptotic limit in ionization energy and consequently reached a saturation point relative to insulation properties at —(CH₂)₉ or insertion of a n-nonyl spacer between the fluorocarbon chain and the P atom.

As with any large molecule requiring computational treatment there is a strong desire to extract meaningful results at the lowest possible theoretical level. In our study, ionization energies were computed using both the low-end Koopman's theorem approximation at the HF/3-21G* and HF/3-21+G* levels of theory, as well as VIPs determined at the computationally more rigorous density functional approximation with the B3LYP functional. All neutral ligands were optimized at the B3LYP/6-31+G* level of theory, with a second calculation required at this geometry for the ligand cation. In addition, a single point calculation using B3LYP/6-31+G** was carried out at the B3LYP/6-31+G* optimized ground state neutral ligand geometry.

TABLE 3
Calculated Vertical Ionization Potentials for 1-4 and a related molecule

| | | VIP (eV) |
|---|---|---|
| Substituent | | (HF/3-21G*) |
| —(CH₂)₃CF₃ | 1 | 8.81 |
| —(CH₂)₃CF₂CF₃ | | 8.84 |
| —(CH₂)₃(CF₂)₅CF₃ | 2 | 8.88 |
| —(CH₂)₃(CF₂)₇CF₃ | 3 | 8.88 |
| —(CH₂)₃(CF₂)₉CF₃ | 4 | 8.88 |

When the electrochemical data are considered in combination with the theoretical results three conclusions can be made.

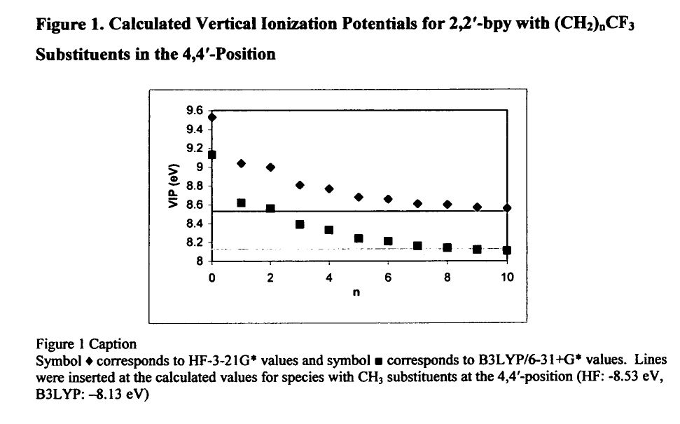

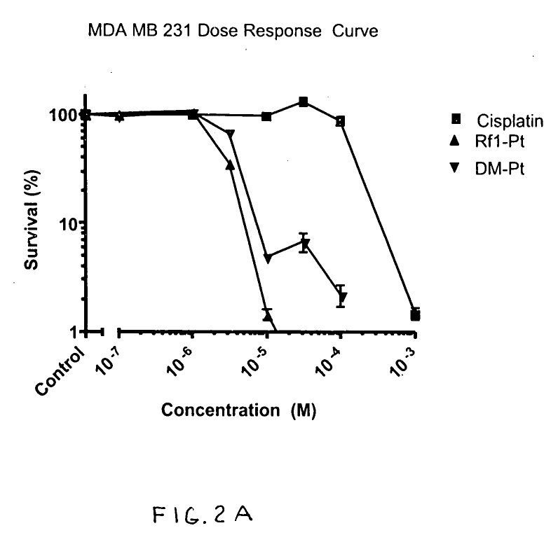

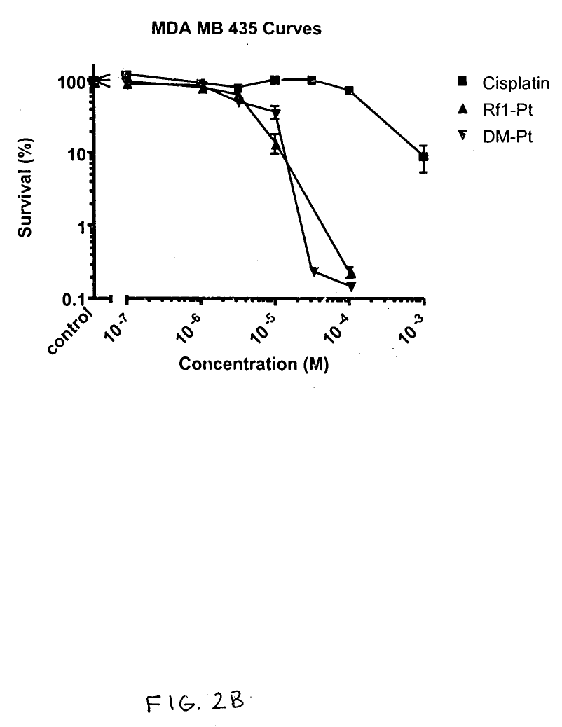

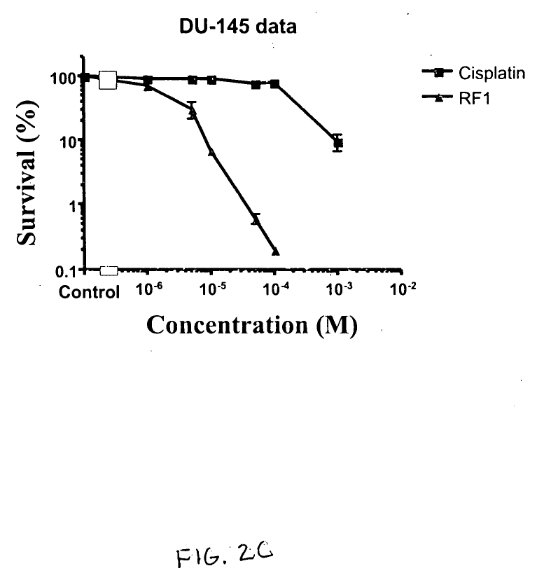

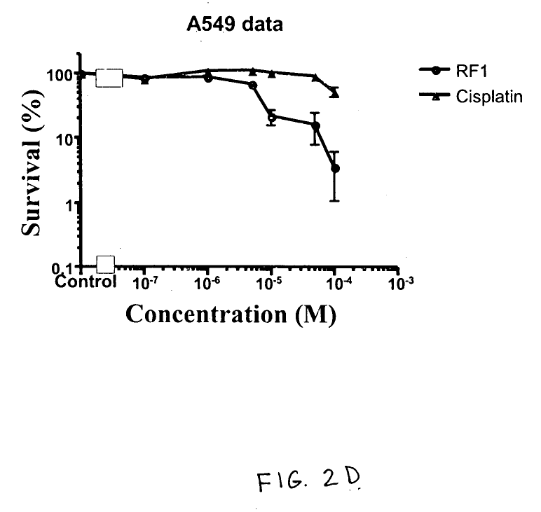

FIG. 1 shows a graphic representation of Calculated Vertical Ionization Potentials for 2,2′-bpy with (CH₂)ₙCF₃ Substituents in the 4,4′-Position. The Symbol ♦ corresponds to HF-3-21G* values and symbol U corresponds to B3LYP/6-31+G* values. Lines were inserted at the calculated values for species with CH₃ substituents at the 4,4′-position (HF: −8.53 eV, B3LYP: −8.13 eV)

First, complete insulation of the perfluorinated segment of the alkyl substituent from the 2,2′-bipyridine moiety is not accomplished without insertion of 9-10 methylene “spacers” as seen in FIG. 1. VIP values similar to 4,4′-dimethyl-2,2′-bipyridine are not observed until n=9 (B3LYP/6-31+G*) and n=10 (HF-3-21G*). Further the lower level calculations provided a similar trend supporting the method's validity for the system.

Second, a distinct limit of electronic impact is found for extension of the perfluoroalkyl chain while holding the spacer size constant (n-propyl, table 3). The limit is found between —(CF₂)₂CF₃ and —(CF₂)₄CF₃ as 2, 3, 4 all exhibit identical VIPs in theory and experimentally 2 and 3 do in fact exhibit similar reduction potentials.

Third, from the calculated VIPs and experimental reduction potentials one can see that the three methylene spacers (n-propyl) serve to attenuate the impact of the perfluoroalkyl group incompletely, affording reduction potentials that are not equal to methyl or n-nonyl substituents but are almost equivalent to a hydrogen substituent (or unsubstituted 2,2′-bipyridine) thus H≅(CH₂)₃(CF₂)ₓ₋₁CF₃ (x=1-10). Previous observations for coordinated Cp systems support this conclusion.^{18 19 vii} The principle is illustrated well in terms of the reduction potentials above. The carbon skeletons of the n-nonyl substituted derivative and 2 are identical, however the reduction potential of 2 is 94 mV less than the alkyl analogue (similar to the observed difference between unsubstituted 2,2′-bipyridine (4,4′-H) and 4,4′-dimethyl-2,2′-bipyridine: 88 mV).

Design of ligands for support of transition metal catalysts intended for use in FBS applications often requires a careful tuning of substituents. Modification of aromatic compounds without impacting the electronic character of the original molecule can be accomplished by implementing three methylenes as insulators between the R_{fx} group and the aromatic moiety. Biochemical experiments have been limited to invitro tests, but have been shown to be effective and the rates of activity and cytoxicity have been evaluated.

Experimental Section

### Preparation of 4,4′-bis(4,4,4-trifluorobutyl)-2,2′-bipyridine, 1

A three-neck 250 mL round bottomed flask was charged with 2.475 g [24.46 mmol] of diisopropylamine dissolved in 100 mL of THF. The ambient atmosphere in the flask was replaced with nitrogen and was cooled to −78° C. Addition of 8.78 mL of n-BuLi in hexanes (2.541M) [21.52 mmol] was completed over a 5-minute period via syringe. After 20 minutes the reaction mixture was warmed to 0° C., stirred for 30 minutes, then cooled to −78° C. 1.992 g [10.81 mmol] of 4,4′-dimethyl-2,2′-bipyridine, dissolved in 20 mL of THF, was added via syringe and stirring was continued for 3 hours after which 4.863 g [22.11 mmol] of 3,3,3-trifluoro-1-iodopropane dissolved in 50 mL of THF was added via syringe. After an hour the reaction mixture was allowed to warm slowly to ambient temperature overnight. The reaction was quenched with 150 mL of brine; the residue was extracted into Et₂O and dried over Na₂SO₄. Evaporation afforded crude product. After recrystallization from MeOH, 2.15 g [5.71 mmol] was isolated as a white solid (52.8% yield). Analytical Calculated for C₁₈H₁₈F₆N₂: C, 57.45; H, 4.82; N, 7.44. Found: C, 57.73; H, 4.74; N, 7.53.

| Cell line | Cisplatin | 1 | DM-Pt |
|---|---|---|---|
| MDA-MB-435 | 132 ± 2 | 2.2 ± 1.6 * | 4.5 ± 1.1 * |
| MDA-MB-231 | 237 ± 0 | 1.9 ± 0.1 * | 5.1 ± 2.8 * |

IC₅₀ values (nM) for a one hour exposure display a significant difference between cisplatin (P<0.05). The DM-Pt complex is (4,4′-dimethyl-2,2′-bipyridine)PtCl₂. Note that this data confirms enhanced reactivity for two more cell lines not mentioned earlier.

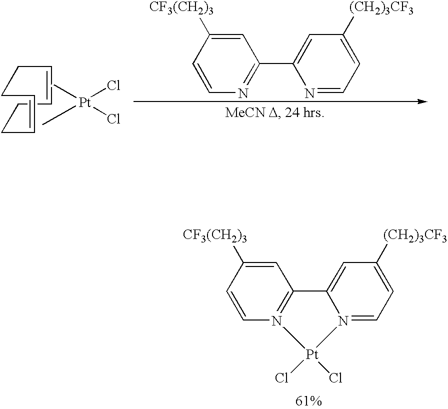

### Preparation of [4,4′-CF₃(CH₂)₃-2,2′-bipyridine]PtCl₂

A 100 mL round bottomed flask was charged with a stir bar, 35 mg (1,5-COD-²η-)PtCl₂ [0.0940 mmole], 39 mg 4,4′-CF₃(CH₂)₃-2,2′-bipyridine [0.1036 mmole], and 15 mL of MeCN. The mixture was brought to reflux for approximately 24 hrs. Upon cooling the solvent was evaporated and residual COD removed via oil pump vac. The residue was then dissolved in MeCN and product precipitated with Et₂O. The yellow solid was recovered in 61% yield, 37 mg [0.0467 mmole].

Elemental Analysis: Calculated for C₁₈H₁₈Cl₂F₆N₂Pt: C, 33.66%; H, 2.82%; N, 4.36%. Found: C, 33.81%; H, 2.80%; N, 4.25%.

¹H NMR (400 MHz, 295K, acetone-d₆) δ 9.513 (d, ³J_{HH}=6.0 Hz, 2H, —CH 6/6′), 8.467(s, 2H, —CH 3/3′), 7.712(d, ³J_{HH}=6.0 Hz, 2H, —CH 5/5′), 2.999(t, ³J_{HH}=8.8 Hz, 4H, —CH₂CH₂CH₂CF₃), 2.379(m, 4H, —CH₂CH₂CH₂CF₃), 2.100(m, 4H, —CH₂CH₂CH₂CF₃);

¹³C{¹H} NMR (295 K, acetone-d₆) δ: 157.874 (CC, 2/2′,2C), 155.944 (CCH₂-, 4/4′, 2C), 149.036 (CH, CH 6/6′, 2C), 128.001 (CH, 3/3′, 2C), 125.030 (5/5′,2C), 128.397 (q, ¹J_{FC}=275.5 Hz, CF₃, 2C), 35.005 (—CH₂CH₂CH₂CF₃,2C) 33.45 (q, ²J_{FC}=28.5 Hz, —CH₂CH₂CH₂CF₃, 2C), 22.700 (q, ³J_{FC}=3.1 Hz, —CH₂CH₂CH₂CF₃, 2C)

¹⁹F NMR: (295 K, acetone-d₆) δ: −66.5 (t, ³J_{HF}=11.2 Hz, —CF₃, 6F)

Quoting: Byron L. Bennett, Kathleen A. Robins, Ryan Tennant, Kyler Elwell, Felice Ferri, Inna Bashta, and Grant Aguinaldo, “Fluorous Modification of 2,2′-Bipyridine”, Journal of Fluorine Chemistry, 2006, 127(1), 140.

[generic description of the preparative route for the 2,2′-bipyridines. The same route generates 1,10-phenanthroline compounds if one uses 4,7-dimethyl-1,10-phenanthroline as a starting material.]

As shown in Scheme 1 the preparation of 2,2′-bipyridines 1-4 features in-situ generation of the 4,4′-dilithioanion of 4,4′-dimethyl-2,2′-bipyridine followed by nucleophilic substitution of iodide from the appropriate fluorous primary alkyl iodide.

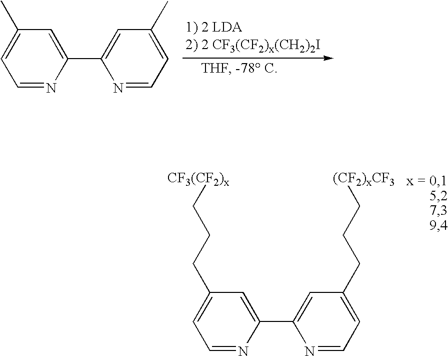

[Specific preparative route to 2,2′-bipyridine derivative]

### Preparation of 4,4′-bis(4,4,4-trifluorobutyl)-2,2′-bipyridine, 1

A three-neck 250 mL round bottomed flask was charged with 2.475 g [24.46 mmol] of diisopropylamine dissolved in 100 mL of THF. The ambient atmosphere in the flask was replaced with nitrogen and was cooled to −78° C. Addition of 8.78 mL of n-BuLi in hexanes (2.541M) [21.52 mmol] was completed over a 5-minute period via syringe. After 20 minutes the reaction mixture was warmed to 0° C., stirred for 30 minutes, then cooled to −78° C. 1.992 g [10.81 mmol] of 4,4′-dimethyl-2,2′-bipyridine, dissolved in 20 mL of THF, was added via syringe and stirring was continued for 3 hours after which 4.863 g [22.11 mmol] of 3,3,3-trifluoro-1-iodopropane dissolved in 50 mL of THF was added via syringe. After an hour the reaction mixture was allowed to warm slowly to ambient temperature overnight. The reaction was quenched with 150 mL of brine; the residue was extracted into Et₂O and dried over Na₂SO₄. Evaporation afforded crude product. After recrystallization from MeOH, 2.15 g [5.71 mmol] was isolated as a white solid (52.8% yield).

Anal. Calcd for C₁₈H₁₈F₆N₂: C, 57.45; H, 4.82; N, 7.44. Found: C, 57.73; H, 4.74; N, 7.53. NMR (298K, CDCl₃): ¹H δ 8.62 (d, ³J_{HH}=5.0 Hz 2H, C—H 6,6′) 8.28 (d, ⁴J_{HH}=1.0 Hz 2H, C—H 3,3′) 7.17 (d,d ³J_{HH}=5.0 Hz, ⁴J_{HH}=1.0 Hz 2H, C—H 5,5′) 2.81 (t, ³J_{HH}=7.7 Hz, 4H, CF₃CH₂CH₂CH₂—Ar) 2.14 (m, 4H, CF₃CH₂CH₂CH₂—Ar) 2.01 (m, 4H, CF₃CH₂CH₂CH₂—Ar); ¹³C{¹H} δ 156.5 (2C, 2/2′) 150.9 (2C, 4/4′) 149.5 (2C, 6/6′) 126.9 (q, ¹J_{FC}=276 Hz, 2C CF₃CH₂CH₂CH₂—Ar) 124.0 (2C, 3/3′) 121.3 (2C, 5/5′) 34.4 (2C CF₃CH₂CH₂CH₂—Ar) 33.3 (q, ²J_{FC}=29 Hz, 2C CF₃CH₂CH₂CH₂—Ar) 22.7 (2C CF₃CH₂CH₂CH₂—Ar); ¹⁹F δ-71.17 (t, 6F, ³J_{HF}=10 Hz); MS (EI: 70 eV, m/z) 376.35 (M⁺, 4%), 357.25 (M⁺-F, 1%), 307.35 (M⁺-CF₃, 6%), 293.25 (M⁺-CH₂CF₃, 13%), 280.30 (M⁺-CH₂CH₂CF₃, 100%), 265.30 (M⁺-CH₂CH₂CH₂CF₃, 2%), UV-Vis (CH₂Cl₂, 25° C.) λ, nm (ε: cm⁻¹M⁻¹) 282.2 (18400), 249.2 (12500), 241.9 (12700). IR (microcrystalline powder, cm⁻¹) v 3057 (w), 3027 (m), 3007 (m), 2958 (s), 2878 (s), 1931 (m), 1599 (s), 1557 (s), 1464 (s), 1397 (s), 1268 (s), 1142 (s), 1070 (m), 1019 (m), 975 (m), 911 (m), 839 (s), 816 (w), 784 (w), 764 (w).

Additional materials and synthetic procedures for known and prophetic ether ligands and platinum complexes include at least the following:

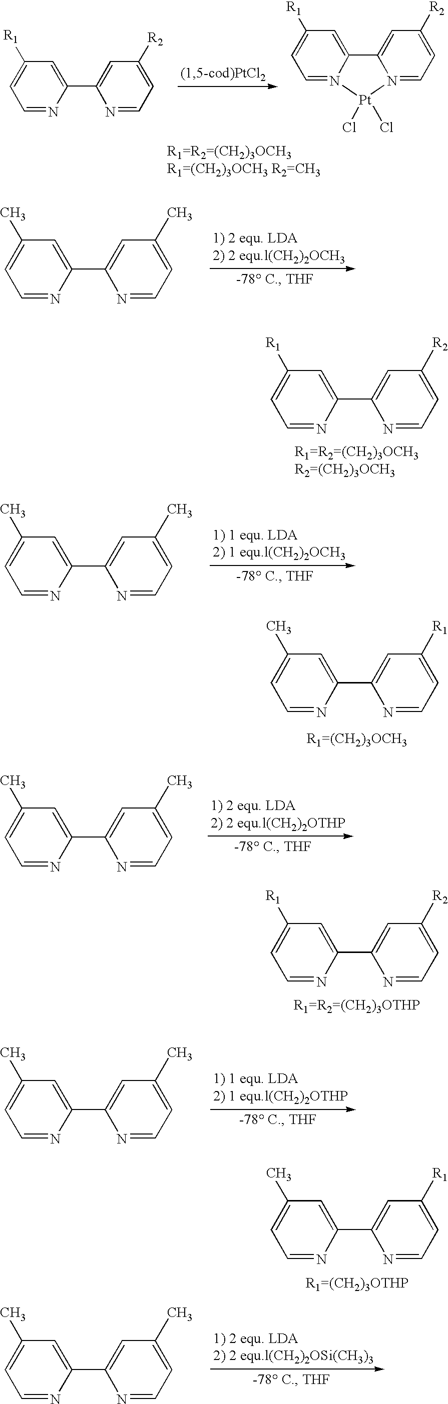

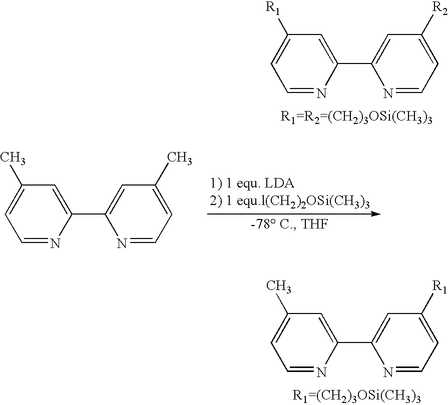

As given in the figure above several ether derivatives should be protected as intellectual property. As support for this assertion, the methyl ether derivatives have been isolated, platinum complexes generated, and cytotoxicity determined in several cell lines. The di substituted methylether derivative has been studied extensively as the elemental analysis, ¹H NMR and ¹³C NMR, and MS all have been acquired for ligand. The platinum complex has been treated in similar fashion with the exception of MS. In all cases the analyses support the composition of the ligand and complex and are of publication quality. The silyl and THP ether derivatives will be studied shortly and are expected to be easy to isolate and characterize.

The cytotoxicity of the initial dimethylether compounds was very similar to the dimethylcomplex and fluorinated derivatives. All of the ether derivatives were generated in an effort to isolate the alcohol derivative shown in the figure below. Protolytic processes will accomplish the cleavage of the ether compounds or complexes. In the case of the silyl ether compounds and complexes TBAF can be utilized as well. The ethers and alcohols represent an important ensemble with regard to binding to the exterior of the major and minor groove of DNA. With regard to the chemical community, the extension of the fluorine substituted derivatives to terminal alcohol derivatives will appear logical and expected to afford greater functionality with regard to binding to the exterior of DNA based on the hydrogen bonding of the polar terminal groups. 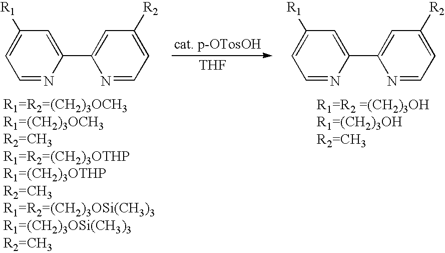
Clearly the broad classification of the derivatives to protect might include those including structures containing alternate lengths of alkyl spacers between the 2,2′-bipyridine or 1,10-phenanthroline ring system (i.e., (CH₂)ₙ where n=3-5) and the functional group terminus. For derivatives longer than 10 methylene units solubility becomes a problem at least for what we currently observe in DMSO which Dr. Carper routinely uses for cytotoxcity surveys. The data indicate that at 10 micromolar application concentrations, cisplatin treated samples of DC4 and DB46 had a survival of about 20%, while the RF1 treated cells had a survival around 2%.

FIG. 1 shows a graphic representation of Calculated Vertical Ionization Potentials for 2,2′-bpy with (CH₂)ₙCF₃ Substituents in the 4,4′-Position. The Symbol ♦ corresponds to HF-3-21G* values and symbol ▪ corresponds to B3LYP/6-31+G* values. Lines were inserted at the calculated values for species with CH₃ substituents at the 4,4′-position (HF: −8.53 eV, B3LYP: −8.13 eV).

Although specific materials, times, temperature and weights are described in the examples, these specific values are intended to be examples supporting the generic concepts described herein, and are not to be interpreted as limiting the generic scope of the disclosure.

Appendix I

Single crystal diffraction data for fac-[4,4′-Bis(4,4,4-trifluorobutyl)-2,2′-bipyridine]Re(CO)₃Cl is provided below as solid state structural detail for 4,4′-Bis(4,4,4-trifluorobutyl)-2,2′-bipyridine coordinated to a low valent transition metal (ex. Re(I)). Similar bond lengths, angles, and torsional angles are expected for solid state detail of the 4,4′-substituted-2,2′-bipyridine complexes of Pt(II) (ie. LPtCl₂).

TABLE 1
Crystal data and structure refinement for vjc456fm.

| | |
|---|---|
| Identification code | vjc456fm |
| Empirical formula | C42H35Cl2F12N4O6Re2 |
| Formula weight | 1363.04 |
| Temperature | 100(2) K |
| Wavelength | 0.71073 Å |
| Crystal system | Triclinic |
| Space group | P-1 |
| Unit cell dimensions | a = 12.2388(4) Å α = 80.2570(10)°. |
| | b = 13.6535(5) Å β = 88.9020(10)°. |
| | c = 14.6184(5) Å γ = 84.1890(10)°. |
| Volume | 2395.16(14) Å³ |
| Z | 2 |
| Density (calculated) | 1.890 Mg/m³ |
| Absorption coefficient | 5.257 mm⁻¹ |
| F(000) | 1310 |
| Crystal size | 0.16 × 0.15 × 0.11 mm³ |
| Theta range for data collection | 1.89 to 27.50°. |
| Index ranges | −15 <= h <= 15, −17 <= k <= 17, |
| | −18 <= 1 <= 18 |
| Reflections collected | 31611 |
| Independent reflections | 10984 [R(int) = 0.0266] |
| Completeness to theta = 27.50° | 99.9% |
| Absorption correction | SADABS |
| Max. and min. transmission | 0.5981 and 0.4849 |
| Refinement method | Full-matrix least-squares on F² |
| Data/restraints/parameters | 10984/0/613 |
| Goodness-of-fit on F² | 1.064 |
| Final R indices [I > 2sigma(I)] | R1 = 0.0326, wR2 = 0.0848 |
| R indices (all data) | R1 = 0.0409, wR2 = 0.0881 |
| Largest diff. peak and hole | 3.364 and −1.580 e.Å⁻³ |

TABLE 2
Atomic coordinates (×10⁴) and equivalent isotropic
displacement parameters (Å² × 10³) for vjc456fm.
U(eq) is defined as one third of the trace of the orthogonalized U^{ij} tensor.

| | x | y | z | U(eq) |
|---|---|---|---|---|
| Re(1) | 5123(1) | 5351(1) | 2756(1) | 27(1) |
| Cl(1) | 6654(1) | 4430(1) | 2023(1) | 33(1) |
| O(1) | 6845(3) | 6470(3) | 3549(3) | 45(1) |
| O(2) | 5191(3) | 3867(3) | 4590(2) | 36(1) |
| O(3) | 3193(3) | 6512(3) | 3583(3) | 47(1) |
| N(1) | 4095(3) | 4598(3) | 1957(3) | 27(1) |
| N(2) | 4933(3) | 6303(3) | 1407(3) | 28(1) |
| F(1) | −1044(7) | 2369(8) | 1008(6) | 170(4) |
| F(2) | −1461(5) | 2366(6) | −412(6) | 150(3) |
| F(3) | −1318(5) | 3736(6) | 54(8) | 195(5) |
| F(4) | 1453(3) | 10392(3) | −1408(3) | 67(1) |
| F(5) | 2185(3) | 11354(3) | −2502(2) | 59(1) |
| F(6) | 2831(3) | 11119(3) | −1121(3) | 55(1) |
| C(1) | 6163(5) | 6067(4) | 3277(3) | 34(1) |
| C(2) | 5181(4) | 4422(3) | 3895(3) | 29(1) |
| C(3) | 3920(5) | 6076(4) | 3275(3) | 35(1) |
| C(4) | 3725(4) | 3709(4) | 2264(3) | 33(1) |
| C(5) | 3103(4) | 3241(4) | 1729(4) | 35(1) |
| C(6) | 2850(4) | 3690(4) | 818(4) | 34(1) |
| C(7) | 3243(4) | 4603(3) | 498(3) | 30(1) |
| C(8) | 3854(4) | 5047(3) | 1075(3) | 27(1) |
| C(9) | 4299(4) | 6012(3) | 765(3) | 25(1) |
| C(10) | 4087(4) | 6586(3) | −101(3) | 27(1) |
| C(11) | 4535(4) | 7486(3) | −342(3) | 30(1) |
| C(12) | 5197(4) | 7766(4) | 310(3) | 33(1) |
| C(13) | 5374(4) | 7177(3) | 1160(3) | 33(1) |
| C(14) | 2173(5) | 3208(4) | 199(4) | 45(1) |
| C(15) | 980(5) | 3278(6) | 481(6) | 65(2) |
| C(16) | 316(6) | 2767(6) | −138(7) | 75(2) |
| C(17) | −896(8) | 2765(10) | 189(10) | 108(4) |
| C(18) | 4264(5) | 8158(4) | −1259(3) | 34(1) |
| C(19) | 3443(4) | 9031(4) | −1089(3) | 33(1) |
| C(20) | 3169(5) | 9792(4) | −1964(3) | 37(1) |
| C(21) | 2414(5) | 10656(4) | −1758(4) | 40(1) |
| Re(2) | −2840(1) | −1582(1) | 5265(1) | 24(1) |
| Cl(2) | −1553(1) | −1584(1) | 6562(1) | 30(1) |
| O(4) | −2695(3) | −3876(3) | 5636(3) | 40(1) |
| O(5) | −4853(3) | −1377(3) | 6530(2) | 36(1) |
| O(6) | −4436(3) | −1514(3) | 3666(2) | 37(1) |
| N(3) | −2620(3) | −2(3) | 4914(3) | 26(1) |
| N(4) | −1362(3) | −1518(3) | 4423(3) | 29(1) |
| F(7) | 853(4) | 4396(4) | 2879(6) | 133(3) |
| F(8) | −381(5) | 4706(4) | 1828(4) | 103(2) |
| F(9) | −39(4) | 5830(3) | 2624(4) | 89(2) |
| F(10) | 2887(5) | 806(5) | 1337(5) | 149(3) |
| F(11) | 3710(4) | −211(5) | 2338(3) | 102(2) |
| F(12) | 4244(3) | −132(3) | 943(2) | 56(1) |
| C(31) | −997(4) | −602(3) | 4161(3) | 28(1) |
| C(28) | −2191(4) | 2009(3) | 4437(3) | 30(1) |
| C(25) | −3815(4) | −1544(3) | 4259(3) | 27(1) |
| C(24) | −4095(4) | −1474(3) | 6064(3) | 28(1) |
| C(32) | −28(4) | −483(4) | 3672(3) | 33(1) |
| C(29) | −1481(4) | 1240(3) | 4177(3) | 30(1) |
| C(30) | −1706(4) | 247(3) | 4420(3) | 26(1) |
| C(35) | −757(4) | −2311(4) | 4191(3) | 34(1) |
| C(26) | −3300(4) | 742(4) | 5169(3) | 32(1) |
| C(23) | −2781(4) | −3020(4) | 5505(3) | 31(1) |
| C(34) | 222(4) | −2232(4) | 3697(4) | 36(1) |
| C(33) | 602(4) | −1304(4) | 3445(4) | 37(1) |
| C(36) | −1999(5) | 3101(4) | 4202(4) | 39(1) |
| C(27) | −3118(4) | 1735(4) | 4940(3) | 33(1) |
| C(38) | −953(5) | 4498(4) | 3403(4) | 49(1) |
| C(43) | 3362(5) | −120(5) | 1505(4) | 44(1) |
| C(40) | 1681(5) | −1170(5) | 2920(4) | 49(1) |
| C(37) | −1036(5) | 3366(4) | 3568(5) | 51(2) |
| C(39) | −107(6) | 4849(5) | 2723(7) | 70(2) |
| C(41) | 1492(7) | −733(9) | 1932(5) | 100(4) |
| C(42) | 2578(8) | −784(10) | 1331(7) | 129(5) |

TABLE 3
Bond lengths [Å] and angles [°] for vjc456fm.

| | | | |
|---|---|---|---|
| Re(1)—C(3) | 1.907(5) | N(4)—C(35) | 1.336(6) |
| Re(1)—C(2) | 1.912(4) | N(4)—C(31) | 1.362(6) |
| Re(1)—C(1) | 1.924(5) | F(7)—C(39) | 1.276(8) |
| Re(1)—N(2) | 2.173(4) | F(8)—C(39) | 1.407(10) |
| Re(1)—N(1) | 2.174(4) | F(9)—C(39) | 1.333(8) |
| Re(1)—Cl(1) | 2.4791(12) | F(10)—C(43) | 1.323(8) |
| O(1)—C(1) | 1.155(6) | F(11)—C(43) | 1.279(7) |
| O(2)—C(2) | 1.161(5) | F(12)—C(43) | 1.345(6) |
| O(3)—C(3) | 1.148(6) | C(31)—C(32) | 1.383(6) |
| N(1)—C(4) | 1.342(6) | C(31)—C(30) | 1.474(6) |
| N(1)—C(8) | 1.355(6) | C(28)—C(27) | 1.389(7) |
| N(2)—C(13) | 1.349(6) | C(28)—C(29) | 1.393(7) |
| N(2)—C(9) | 1.363(6) | C(28)—C(36) | 1.514(7) |
| F(1)—C(17) | 1.245(15) | C(32)—C(33) | 1.378(7) |
| F(2)—C(17) | 1.346(11) | C(29)—C(30) | 1.395(6) |
| F(3)—C(17) | 1.357(14) | C(35)—C(34) | 1.391(7) |
| F(4)—C(21) | 1.333(6) | C(26)—C(27) | 1.380(7) |
| F(5)—C(21) | 1.332(6) | C(34)—C(33) | 1.384(7) |
| F(6)—C(21) | 1.345(6) | C(33)—C(40) | 1.526(7) |
| C(4)—C(5) | 1.374(7) | C(36)—C(37) | 1.521(8) |
| C(5)—C(6) | 1.395(7) | C(38)—C(39) | 1.480(10) |
| C(6)—C(7) | 1.385(6) | C(38)—C(37) | 1.536(7) |
| C(6)—C(14) | 1.510(7) | C(43)—C(42) | 1.440(10) |
| C(7)—C(8) | 1.388(6) | C(40)—C(41) | 1.480(10) |
| C(8)—C(9) | 1.474(6) | C(41)—C(42) | 1.581(10) |
| C(9)—C(10) | 1.385(6) | C(3)—Re(1)—C(2) | 87.3(2) |
| C(10)—C(11) | 1.385(6) | C(3)—Re(1)—C(1) | 91.5(2) |
| C(11)—C(12) | 1.387(7) | C(2)—Re(1)—C(1) | 88.2(2) |
| C(11)—C(18) | 1.513(6) | C(3)—Re(1)—N(2) | 92.70(18) |
| C(12)—C(13) | 1.370(7) | C(2)—Re(1)—N(2) | 173.63(17) |
| C(14)—C(15) | 1.507(8) | C(1)—Re(1)—N(2) | 98.16(17) |
| C(15)—C(16) | 1.524(10) | C(3)—Re(1)—N(1) | 94.63(19) |
| C(16)—C(17) | 1.550(12) | C(2)—Re(1)—N(1) | 99.05(17) |
| C(18)—C(19) | 1.530(7) | C(1)—Re(1)—N(1) | 170.67(18) |
| C(19)—C(20) | 1.524(6) | N(2)—Re(1)—N(1) | 74.59(14) |
| C(20)—C(21) | 1.493(8) | C(3)—Re(1)—Cl(1) | 177.83(14) |
| Re(2)—C(25) | 1.903(5) | C(2)—Re(1)—Cl(1) | 94.49(14) |
| Re(2)—C(24) | 1.920(5) | C(1)—Re(1)—Cl(1) | 89.72(15) |
| Re(2)—C(23) | 1.929(5) | N(2)—Re(1)—Cl(1) | 85.37(11) |
| Re(2)—N(4) | 2.168(4) | N(1)—Re(1)—Cl(1) | 83.91(10) |
| Re(2)—N(3) | 2.174(4) | C(4)—N(1)—C(8) | 118.2(4) |
| Re(2)—Cl(2) | 2.4869(11) | C(4)—N(1)—Re(1) | 124.6(3) |
| O(4)—C(23) | 1.146(6) | C(8)—N(1)—Re(1) | 117.1(3) |
| O(5)—C(24) | 1.151(6) | C(13)—N(2)—C(9) | 117.2(4) |
| O(6)—C(25) | 1.157(6) | C(13)—N(2)—Re(1) | 125.0(3) |
| N(3)—C(26) | 1.345(6) | C(9)—N(2)—Re(1) | 117.8(3) |
| N(3)—C(30) | 1.361(6) | O(1)—C(1)—Re(1) | 175.0(5) |
| O(3)—C(3)—Re(1) | 179.7(5) | O(2)—C(2)—Re(1) | 178.4(4) |
| N(1)—C(4)—C(5) | 123.0(4) | N(4)—Re(2)—Cl(2) | 83.79(11) |
| C(4)—C(5)—C(6) | 119.6(4) | N(3)—Re(2)—Cl(2) | 84.95(10) |
| C(7)—C(6)—C(5) | 117.4(4) | C(26)—N(3)—C(30) | 117.7(4) |
| C(7)—C(6)—C(14) | 120.9(5) | C(26)—N(3)—Re(2) | 125.1(3) |
| C(5)—C(6)—C(14) | 121.7(5) | C(30)—N(3)—Re(2) | 117.2(3) |
| C(6)—C(7)—C(8) | 120.5(4) | C(35)—N(4)—C(31) | 118.1(4) |
| N(1)—C(8)—C(7) | 121.3(4) | C(35)—N(4)—Re(2) | 125.0(3) |
| N(1)—C(8)—C(9) | 116.1(4) | C(31)—N(4)—Re(2) | 116.8(3) |
| C(7)—C(8)—C(9) | 122.5(4) | N(4)—C(31)—C(32) | 121.6(4) |
| N(2)—C(9)—C(10) | 122.3(4) | N(4)—C(31)—C(30) | 115.8(4) |
| N(2)—C(9)—C(8) | 114.4(4) | C(32)—C(31)—C(30) | 122.5(4) |
| C(10)—C(9)—C(8) | 123.3(4) | C(27)—C(28)—C(29) | 116.8(4) |
| C(9)—C(10)—C(11) | 119.9(4) | C(27)—C(28)—C(36) | 119.6(4) |
| C(10)—C(11)—C(12) | 117.2(4) | C(29)—C(28)—C(36) | 123.6(5) |
| C(10)—C(11)—C(18) | 121.1(4) | O(6)—C(25)—Re(2) | 177.8(4) |
| C(12)—C(11)—C(18) | 121.6(4) | O(5)—C(24)—Re(2) | 177.8(4) |
| C(13)—C(12)—C(11) | 120.6(4) | C(33)—C(32)—C(31) | 120.1(5) |
| N(2)—C(13)—C(12) | 122.7(4) | C(28)—C(29)—C(30) | 120.7(4) |
| C(15)—C(14)—C(16) | 111.2(5) | N(3)—C(30)—C(29) | 121.5(4) |
| C(14)—C(15)—C(16) | 110.3(6) | N(3)—C(30)—C(31) | 115.0(4) |
| C(15)—C(16)—C(17) | 110.9(7) | C(29)—C(30)—C(31) | 123.5(4) |
| F(1)—C(17)—F(3) | 110.1(11) | N(4)—C(35)—C(34) | 122.6(5) |
| F(1)—C(17)—F(2) | 112.0(10) | N(3)—C(26)—C(27) | 123.1(4) |
| F(3)—C(17)—F(2) | 103.4(10) | O(4)—C(23)—Re(2) | 176.8(4) |
| F(1)—C(17)—C(16) | 115.9(11) | C(33)—C(34)—C(35) | 119.3(5) |
| F(3)—C(17)—C(16) | 105.9(10) | C(32)—C(33)—C(34) | 118.3(4) |
| F(2)—C(17)—C(16) | 108.6(9) | C(32)—C(33)—C(40) | 119.9(5) |
| C(11)—C(18)—C(19) | 109.0(4) | C(34)—C(33)—C(40) | 121.8(5) |
| C(20)—C(19)—C(18) | 113.3(4) | C(28)—C(36)—C(37) | 117.3(4) |
| C(21)—C(20)—C(19) | 111.5(4) | C(26)—C(27)—C(28) | 120.4(5) |
| F(5)—C(21)—F(4) | 106.5(5) | C(39)—C(38)—C(37) | 115.2(6) |
| F(5)—C(21)—F(6) | 106.4(5) | F(11)—C(43)—F(10) | 103.4(6) |
| F(4)—C(21)—F(6) | 105.0(5) | F(11)—C(43)—F(12) | 107.7(5) |
| F(5)—C(21)—C(20) | 113.2(5) | F(10)—C(43)—F(12) | 105.6(5) |
| F(4)—C(21)—C(20) | 113.0(5) | F(11)—C(43)—C(42) | 117.1(7) |
| F(6)—C(21)—C(20) | 112.3(4) | F(10)—C(43)—C(42) | 108.5(8) |
| C(25)—Re(2)—C(24) | 88.60(19) | F(12)—C(43)—C(42) | 113.5(5) |
| C(25)—Re(2)—C(23) | 89.7(2) | C(41)—C(40)—C(33) | 111.6(5) |
| C(24)—Re(2)—C(23) | 91.11(19) | C(36)—C(37)—C(38) | 110.8(5) |
| C(25)—Re(2)—N(4) | 95.56(16) | F(7)—C(39)—F(9) | 108.7(6) |
| C(24)—Re(2)—N(4) | 172.11(17) | F(7)—C(39)—F(8) | 105.3(8) |
| C(23)—Re(2)—N(4) | 95.60(17) | F(9)—C(39)—F(8) | 103.5(6) |
| C(25)—Re(2)—N(3) | 93.49(17) | F(7)—C(39)—C(38) | 114.8(7) |
| C(24)—Re(2)—N(3) | 98.12(17) | F(9)—C(39)—C(38) | 113.1(7) |
| C(23)—Re(2)—N(3) | 170.29(16) | F(8)—C(39)—C(38) | 110.5(6) |
| N(4)—Re(2)—N(3) | 74.98(14) | C(40)—C(41)—C(42) | 112.5(8) |
| C(25)—Re(2)—Cl(2) | 178.41(14) | C(43)—C(42)—C(41) | 114.8(7) |
| C(24)—Re(2)—Cl(2) | 91.87(14) | | |
| C(23)—Re(2)—C(2) | 91.77(14) | | |

Symmetry transformations used to generate equivalent atoms:

TABLE 4
Anisotropic displacement parameters (Å² × 10³) for vjc456fm.
The anisotropic displacement factor exponent takes the form:
−2π²[h² a*²U¹¹ + . . . + 2 h k a* b* U¹²]

| | U¹¹ | U²² | U³³ | U²³ | U¹³ | U¹² |
|---|---|---|---|---|---|---|
| Re(1) | 41(1) | 22(1) | 17(1) | −6(1) | −3(1) | 1(1) |
| Cl(1) | 41(1) | 34(1) | 23(1) | −10(1) | −2(1) | 2(1) |
| O(1) | 57(2) | 40(2) | 41(2) | −18(2) | −15(2) | −3(2) |
| O(2) | 43(2) | 39(2) | 23(2) | −2(1) | 0(1) | 6(2) |
| O(3) | 56(2) | 47(2) | 35(2) | −9(2) | −1(2) | 18(2) |
| N(1) | 36(2) | 22(2) | 23(2) | −4(2) | 2(2) | 0(2) |
| N(2) | 39(2) | 24(2) | 24(2) | −8(2) | −1(2) | −6(2) |
| F(1) | 145(6) | 249(10) | 157(7) | −92(7) | 90(6) | −136(7) |
| F(2) | 74(4) | 195(7) | 221(8) | −123(6) | 13(4) | −55(4) |
| F(3) | 59(4) | 157(7) | 403(15) | −153(9) | −13(6) | 1(4) |
| F(4) | 41(2) | 66(2) | 86(3) | 7(2) | 7(2) | −3(2) |
| F(5) | 73(2) | 49(2) | 46(2) | 10(2) | −6(2) | 12(2) |
| F(6) | 66(2) | 44(2) | 58(2) | −20(2) | −22(2) | 7(2) |
| C(1) | 52(3) | 25(2) | 24(2) | −5(2) | −4(2) | 6(2) |
| C(2) | 36(2) | 26(2) | 22(2) | −6(2) | −3(2) | 4(2) |
| C(3) | 52(3) | 27(2) | 25(2) | −5(2) | −8(2) | 9(2) |
| C(4) | 46(3) | 27(2) | 24(2) | −1(2) | 6(2) | −4(2) |
| C(5) | 45(3) | 23(2) | 39(3) | −3(2) | 6(2) | −11(2) |
| C(6) | 39(3) | 27(2) | 37(3) | −10(2) | 1(2) | −6(2) |
| C(7) | 38(3) | 27(2) | 26(2) | −4(2) | −2(2) | −6(2) |
| C(8) | 36(2) | 23(2) | 21(2) | −6(2) | 2(2) | 0(2) |
| C(9) | 31(2) | 22(2) | 22(2) | −7(2) | 0(2) | −2(2) |
| C(10) | 38(2) | 24(2) | 22(2) | −9(2) | −1(2) | −2(2) |
| C(11) | 42(3) | 28(2) | 18(2) | −2(2) | 3(2) | −3(2) |
| C(12) | 46(3) | 25(2) | 28(2) | −5(2) | 2(2) | −7(2) |
| C(13) | 47(3) | 25(2) | 29(2) | −8(2) | −6(2) | −6(2) |
| C(14) | 48(3) | 40(3) | 49(3) | −7(2) | −5(3) | −16(2) |
| C(15) | 48(4) | 72(5) | 86(5) | −38(4) | 12(4) | −17(3) |
| C(16) | 48(4) | 78(5) | 109(7) | −38(5) | 1(4) | −13(4) |
| C(17) | 65(6) | 118(9) | 166(12) | −79(9) | −8(7) | −29(6) |
| C(18) | 54(3) | 29(2) | 19(2) | −2(2) | −3(2) | −7(2) |
| C(19) | 39(3) | 33(3) | 27(2) | 2(2) | −1(2) | −11(2) |
| C(20) | 47(3) | 37(3) | 25(2) | −2(2) | 1(2) | −5(2) |
| C(21) | 40(3) | 45(3) | 35(3) | −1(2) | −4(2) | −7(2) |
| Re(2) | 23(1) | 26(1) | 25(1) | −6(1) | 4(1) | −3(1) |
| Cl(2) | 28(1) | 31(1) | 31(1) | −2(1) | −1(1) | −5(1) |
| O(4) | 47(2) | 31(2) | 42(2) | −5(2) | 4(2) | −9(2) |
| O(5) | 33(2) | 47(2) | 29(2) | −6(2) | 6(1) | −5(2) |
| O(6) | 33(2) | 46(2) | 31(2) | −8(2) | −1(2) | −1(2) |
| N(3) | 22(2) | 29(2) | 25(2) | −5(2) | 2(1) | 0(2) |
| N(4) | 26(2) | 28(2) | 33(2) | −8(2) | 4(2) | 0(2) |
| F(7) | 53(3) | 81(4) | 243(8) | 37(4) | −5(4) | −8(3) |
| F(8) | 129(5) | 96(4) | 93(4) | −27(3) | 37(3) | −40(3) |
| F(9) | 108(4) | 43(2) | 118(4) | −5(2) | 5(3) | −33(2) |
| F(10) | 111(5) | 108(5) | 186(7) | 62(4) | 81(5) | 38(4) |
| F(11) | 59(3) | 204(6) | 51(2) | −22(3) | 7(2) | −54(3) |
| F(12) | 57(2) | 63(2) | 57(2) | −27(2) | 32(2) | −30(2) |
| C(31) | 27(2) | 30(2) | 26(2) | −3(2) | −1(2) | −3(2) |
| C(28) | 36(2) | 25(2) | 28(2) | −3(2) | −7(2) | 2(2) |
| C(25) | 27(2) | 29(2) | 25(2) | −7(2) | 7(2) | −2(2) |
| C(24) | 31(2) | 31(2) | 24(2) | −7(2) | 0(2) | −5(2) |
| C(32) | 29(2) | 30(2) | 38(3) | −2(2) | 5(2) | −4(2) |
| C(29) | 30(2) | 30(2) | 31(2) | −3(2) | −3(2) | −3(2) |
| C(30) | 25(2) | 29(2) | 25(2) | −5(2) | −1(2) | −1(2) |
| C(35) | 36(3) | 31(2) | 33(3) | −5(2) | 5(2) | 0(2) |
| C(26) | 29(2) | 34(3) | 31(2) | −7(2) | 2(2) | 4(2) |
| C(23) | 29(2) | 35(3) | 30(2) | −9(2) | 5(2) | −6(2) |
| C(34) | 32(2) | 34(3) | 40(3) | −9(2) | 8(2) | 6(2) |
| C(33) | 29(2) | 44(3) | 36(3) | −4(2) | 8(2) | 0(2) |
| C(36) | 47(3) | 27(2) | 42(3) | −7(2) | 8(2) | −1(2) |
| C(27) | 35(3) | 34(3) | 30(2) | −7(2) | −3(2) | 5(2) |
| C(38) | 57(4) | 31(3) | 57(4) | −3(3) | −13(3) | −7(3) |
| C(43) | 46(3) | 52(3) | 38(3) | −14(3) | 17(2) | −11(3) |
| C(40) | 40(3) | 43(3) | 60(4) | −1(3) | 16(3) | 3(2) |
| C(37) | 38(3) | 35(3) | 78(4) | −8(3) | −7(3) | 1(2) |
| C(39) | 53(4) | 45(4) | 107(7) | 2(4) | −3(4) | −11(3) |
| C(41) | 64(5) | 202(11) | 50(4) | −42(6) | 23(4) | −65(6) |
| C(42) | 99(7) | 227(13) | 101(7) | −101(8) | 73(6) | −106(8) |

TABLE 5
Hydrogen coordinates (×10⁴) and isotropic displacement
parameters (Å² × 10³) for vjc456fm.

| | x | y | z | U(eq) |
|---|---|---|---|---|
| H(4A) | 3900 | 3387 | 2880 | 40 |
| H(5A) | 2848 | 2616 | 1977 | 43 |
| H(7A) | 3092 | 4929 | −121 | 36 |
| H(10A) | 3636 | 6362 | −529 | 33 |
| H(12A) | 5530 | 8373 | 165 | 40 |
| H(13A) | 5826 | 7392 | 1594 | 40 |
| H(14A) | 2242 | 3543 | −453 | 54 |
| H(14B) | 2456 | 2497 | 236 | 54 |
| H(15A) | 689 | 3988 | 430 | 78 |
| H(15B) | 910 | 2955 | 1136 | 78 |
| H(16A) | 643 | 2071 | −123 | 90 |
| H(16B) | 344 | 3122 | −786 | 90 |
| H(18A) | 3943 | 7776 | −1688 | 41 |
| H(18B) | 4941 | 8413 | −1546 | 41 |
| H(19A) | 2757 | 8766 | −832 | 40 |
| H(19B) | 3750 | 9371 | −621 | 40 |
| H(20A) | 3856 | 10035 | −2242 | 44 |
| H(20B) | 2820 | 9465 | −2422 | 44 |
| H(32A) | 205 | 165 | 3492 | 39 |
| H(29A) | −837 | 1392 | 3831 | 36 |
| H(35A) | −1006 | −2952 | 4370 | 40 |
| H(26A) | −3935 | 577 | 5523 | 38 |
| H(34A) | 625 | −2810 | 3535 | 43 |
| H(36A) | −2462 | 3600 | 4442 | 47 |
| H(27A) | −3630 | 2234 | 5128 | 40 |
| H(38A) | −1676 | 4842 | 3187 | 59 |
| H(38B) | −796 | 4698 | 4003 | 59 |
| H(40A) | 2109 | −729 | 3214 | 59 |
| H(40B) | 2119 | −1827 | 2962 | 59 |
| H(37A) | −1131 | 3144 | 2967 | 61 |
| H(37B) | −347 | 3014 | 3854 | 61 |
| H(41A) | 1188 | −27 | 1887 | 120 |
| H(41B) | 942 | −1095 | 1674 | 120 |
| H(42A) | 2935 | −1477 | 1452 | 154 |
| H(42B) | 2375 | −628 | 666 | 154 |

TABLE 6
Torsion angles [°] for vjc456fm.

| | | | |
|---|---|---|---|
| C(3)—Re(1)—N(1)—C(4) | −91.1(4) | C(13)—N(2)—C(9)—C(8) | 179.3(4) |
| C(2)—Re(1)—N(1)—C(4) | −3.1(4) | Re(1)—N(2)—C(9)—C(8) | −2.2(5) |
| C(1)—Re(1)—N(1)—C(4) | 137.7(10) | N(1)—C(8)—C(9)—N(2) | 2.4(6) |
| N(2)—Re(1)—N(1)—C(4) | 177.4(4) | C(7)—C(8)—C(9)—N(2) | −176.3(4) |
| Cl(1)—Re(1)—N(1)—C(4) | 90.5(4) | N(1)—C(8)—C(9)—C(10) | −177.0(4) |
| C(3)—Re(1)—N(1)—C(8) | 91.8(3) | C(7)—C(8)—C(9)—C(10) | 4.3(7) |
| C(2)—Re(1)—N(1)—C(8) | 179.8(3) | N(2)—C(9)—C(10)—C(11) | 0.6(7) |
| C(1)—Re(1)—N(1)—C(8) | −39.5(12) | C(8)—C(9)—C(10)—C(11) | 180.0(4) |
| N(2)—Re(1)—N(1)—C(8) | 0.2(3) | C(9)—C(10)—C(11)—C(12) | 0.6(7) |
| Cl(1)—Re(1)—N(1)—C(8) | −86.6(3) | C(9)—C(10)—C(11)—C(18) | −176.3(4) |
| C(3)—Re(1)—N(2)—C(13) | 85.5(4) | C(10)—C(11)—C(12)—C(13) | −1.2(7) |
| C(2)—Re(1)—N(2)—C(13) | 175.5(14) | C(18)—C(11)—C(12)—C(13) | 175.7(5) |
| C(1)—Re(1)—N(2)—C(13) | −6.5(4) | C(9)—N(2)—C(13)—C(12) | 0.7(7) |
| N(1)—Re(1)—N(2)—C(13) | 179.5(4) | Re(1)—N(2)—C(13)—C(12) | −177.7(4) |
| Cl(1)—Re(1)—N(2)—C(13) | −95.5(4) | C(11)—C(12)—C(13)—N(2) | 0.5(8) |
| C(3)—Re(1)—N(2)—C(9) | −92.9(4) | C(7)—C(6)—C(14)—C(15) | −106.4(6) |
| C(2)—Re(1)—N(2)—C(9) | −2.9(17) | C(5)—C(6)—C(14)—C(15) | 73.4(7) |
| C(1)—Re(1)—N(2)—C(9) | 175.1(3) | C(6)—C(14)—C(15)—C(16) | −178.8(6) |
| N(1)—Re(1)—N(2)—C(9) | 1.1(3) | C(14)—C(15)—C(16)—C(17) | 175.8(8) |
| Cl(1)—Re(1)—N(2)—C(9) | 86.1(3) | C(15)—C(16)—C(17)—F(1) | −56.9(12) |
| C(3)—Re(1)—C(1)—O(1) | −167(5) | C(15)—C(16)—C(17)—F(3) | 65.6(12) |
| C(2)—Re(1)—C(1)—O(1) | 106(5) | C(15)—C(16)—C(17)—F(2) | 176.1(9) |
| N(2)—Re(1)—C(1)—O(1) | −74(5) | C(10)—C(11)—C(18)—C(19) | 103.5(5) |
| N(1)—Re(1)—C(1)—O(1) | −35(6) | C(12)—C(11)—C(18)—C(19) | −73.2(6) |
| Cl(1)—Re(1)—C(1)—O(1) | 11(5) | C(11)—C(18)—C(19)—C(20) | 176.3(4) |
| C(3)—Re(1)—C(2)—O(2) | 18(16) | C(18)—C(19)—C(20)—C(21) | −176.9(4) |
| C(1)—Re(1)—C(2)—O(2) | 110(16) | C(19)—C(20)—C(21)—F(5) | 177.9(4) |
| N(2)—Re(1)—C(2)—O(2) | −72(16) | C(19)—C(20)—C(21)—F(4) | −61.0(6) |
| N(1)—Re(1)—C(2)—O(2) | −76(16) | C(19)—C(20)—C(21)—F(6) | 57.4(6) |
| Cl(1)—Re(1)—C(2)—O(2) | −160(16) | C(25)—Re(2)—N(3)—C(26) | 83.8(4) |
| C(2)—Re(1)—C(3)—O(3) | −127(100) | C(24)—Re(2)—N(3)—C(26) | −5.3(4) |
| C(1)—Re(1)—C(3)—O(3) | 145(100) | C(23)—Re(2)—N(3)—C(26) | −167.0(9) |
| N(2)—Re(1)—C(3)—O(3) | 47(100) | N(4)—Re(2)—N(3)—C(26) | 178.6(4) |
| N(1)—Re(1)—C(3)—O(3) | −28(100) | Cl(2)—Re(2)—N(3)—C(26) | −96.5(4) |
| Cl(1)—Re(1)—C(3)—O(3) | 20(100) | C(25)—Re(2)—N(3)—C(30) | −98.1(3) |
| C(8)—N(1)—C(4)—C(5) | −0.8(7) | C(24)—Re(2)—N(3)—C(30) | 172.8(3) |
| Re(1)—N(1)—C(4)—C(5) | −178.0(4) | C(23)—Re(2)—N(3)—C(30) | 11.1(11) |
| N(1)—C(4)—C(5)—C(6) | 1.0(8) | N(4)—Re(2)—N(3)—C(30) | −3.3(3) |
| C(4)—C(5)—C(6)—C(7) | −0.3(8) | Cl(2)—Re(2)—N(3)—C(30) | 81.6(3) |
| C(4)—C(5)—C(6)—C(14) | 179.9(5) | C(25)—Re(2)—N(4)—C(35) | −87.6(4) |
| C(5)—C(6)—C(7)—C(8) | −0.6(7) | C(24)—Re(2)—N(4)—C(35) | 150.8(11) |
| C(14)—C(6)—C(7)—C(8) | 179.3(5) | C(23)—Re(2)—N(4)—C(35) | 2.7(4) |
| C(4)—N(1)—C(8)—C(7) | −0.1(7) | N(3)—Re(2)—N(4)—C(35) | −179.7(4) |
| Re(1)—N(1)—C(8)—C(7) | 177.3(3) | Cl(2)—Re(2)—N(4)—C(35) | 93.9(4) |
| C(4)—N(1)—C(8)—C(9) | −178.8(4) | C(25)—Re(2)—N(4)—C(31) | 96.5(3) |
| Re(1)—N(1)—C(8)—C(9) | −1.4(5) | C(24)—Re(2)—N(4)—C(31) | −25.1(14) |
| C(6)—C(7)—C(8)—N(1) | 0.8(7) | C(23)—Re(2)—N(4)—C(31) | −173.2(3) |
| C(6)—C(7)—C(8)—C(9) | 179.4(4) | N(3)—Re(2)—N(4)—C(31) | 4.4(3) |
| C(13)—N(2)—C(9)—C(10) | −1.3(7) | Cl(2)—Re(2)—N(4)—C(31) | −82.0(3) |
| Re(1)—N(2)—C(9)—C(10) | 177.2(3) | C(35)—N(4)—C(31)—C(32) | −0.2(7) |
| Re(2)—N(4)—C(31)—C(32) | 176.0(4) | F(12)—C(43)—C(42)—C(41) | −175.9(8) |
| C(35)—N(4)—C(31)—C(30) | 178.9(4) | C(40)—C(41)—C(42)—C(43) | −71.1(13) |
| Re(2)—N(4)—C(31)—C(30) | −4.9(5) | | |
| C(24)—Re(2)—C(25)—O(6) | 9(11) | | |
| C(23)—Re(2)—C(25)—O(6) | 100(11) | | |
| N(4)—Re(2)—C(25)—O(6) | −164(11) | | |
| N(3)—Re(2)—C(25)—O(6) | −89(11) | | |
| Cl(2)—Re(2)—C(25)—O(6) | −99(12) | | |
| C(25)—Re(2)—C(24)—O(5) | −74(11) | | |
| C(23)—Re(2)—C(24)—O(5) | −164(11) | | |
| N(4)—Re(2)—C(24)—O(5) | 48(11) | | |
| N(3)—Re(2)—C(24)—O(5) | 19(11) | | |
| Cl(2)—Re(2)—C(24)—O(5) | 105(11) | | |
| N(4)—C(31)—C(32)—C(33) | −0.7(8) | | |
| C(30)—C(31)—C(32)—C(33) | −179.7(5) | | |
| C(27)—C(28)—C(29)—C(30) | −0.1(7) | | |
| C(36)—C(28)—C(29)—C(30) | 179.8(4) | | |
| C(26)—N(3)—C(30)—C(29) | −0.4(6) | | |
| Re(2)—N(3)—C(30)—C(29) | −178.7(3) | | |
| C(26)—N(3)—C(30)—C(31) | −179.9(4) | | |
| Re(2)—N(3)—C(30)—C(31) | 1.8(5) | | |
| C(28)—C(29)—C(30)—N(3) | 0.0(7) | | |
| C(28)—C(29)—C(30)—C(31) | 179.5(4) | | |
| N(4)—C(31)—C(30)—N(3) | 2.0(6) | | |
| C(32)—C(31)—C(30)—N(3) | −178.9(4) | | |
| N(4)—C(31)—C(30)—C(29) | −177.5(4) | | |
| C(32)—C(31)—C(30)—C(29) | 1.7(7) | | |
| C(31)—N(4)—C(35)—C(34) | 0.0(7) | | |
| Re(2)—N(4)—C(35)—C(34) | −175.8(4) | | |
| C(30)—N(3)—C(26)—C(27) | 1.0(7) | | |
| Re(2)—N(3)—C(26)—C(27) | 179.1(3) | | |
| C(25)—Re(2)—C(23)—O(4) | 114(8) | | |
| C(24)—Re(2)—C(23)—O(4) | −158(8) | | |
| N(4)—Re(2)—C(23)—O(4) | 18(8) | | |
| N(3)—Re(2)—C(23)—O(4) | 4(8) | | |
| Cl(2)—Re(2)—C(23)—O(4) | −66(8) | | |
| N(4)—C(35)—C(34)—C(33) | 1.0(8) | | |
| C(31)—C(32)—C(33)—C(34) | 1.7(8) | | |
| C(31)—C(32)—C(33)—C(40) | −178.9(5) | | |
| C(35)—C(34)—C(33)—C(32) | −1.9(8) | | |
| C(35)—C(34)—C(33)—C(40) | 178.8(5) | | |
| C(27)—C(28)—C(36)—C(37) | −173.9(5) | | |
| C(29)—C(28)—C(36)—C(37) | 6.2(8) | | |
| N(3)—C(26)—C(27)—C(28) | −1.1(7) | | |
| C(29)—C(28)—C(27)—C(26) | 0.6(7) | | |
| C(36)—C(28)—C(27)—C(26) | −179.3(5) | | |
| C(32)—C(33)—C(40)—C(41) | −74.2(8) | | |
| C(34)—C(33)—C(40)—C(41) | 105.2(7) | | |
| C(28)—C(36)—C(37)—C(38) | −179.8(5) | | |
| C(39)—C(38)—C(37)—C(36) | −174.4(6) | | |
| C(37)—C(38)—C(39)—F(7) | −52.4(10) | | |
| C(37)—C(38)—C(39)—F(9) | −177.9(6) | | |
| C(37)—C(38)—C(39)—F(8) | 66.5(8) | | |
| C(33)—C(40)—C(41)—C(42) | −168.6(7) | | |
| F(11)—C(43)—C(42)—C(41) | 57.7(13) | | |
| F(10)—C(43)—C(42)—C(41) | −58.8(11) | | |

What is claimed:

1. A cis-LPtCl₂ complex wherein L is a 4,4′-substituted-2,2′-bipyridine or a 4,7-substituted-1,10-phenanthroline compound comprising 2,2′-bipyridines and 1,10-phenanthrolines having alkyl groups appended in both the 4,4′-positions of the bipyridine and both of the 4,7-positions of the phenanthroline.

2. The complex of claim 1 wherein the alkyl group is selected from the group consisting of normal, branched and cyclic alkyl groups, alkyl groups with ether linkages, highly fluorinated alkyl group, highly fluorinated alkyl groups with ether linkages, hydroxyl terminated alkyl groups, hydroxyl-terminated alkyl groups with ether linkages and perfluorinated alkyl groups.

3. The complex of claim 2 represented by a formula selected from the group consisting of (CH₂)ₙ(CH₃); (CH₂)ₙ—O—CH₃; CH₂(CH₂)ₙ(CF₂)ₓ₋₁CF₃(CClF)ₙ(CF₂)ₓ₋₁CF₃; and (CH₂)ₙ(CH₂OH) wherein n=0-5, m=0-3; and x=1-6]

4. The complex of claim 3 wherein L comprises a 4,4′-substituted 2,2′-bipyridine.

5. The complex of claim 3 wherein L comprises a 4,7-substituted-1,10-phenanthroline.

6. The complex of claim 3 wherein the 4 and 4′ substituents are asymmetrical with respect to each other.

7. The complex of claim 3 wherein the 4 and 4′ substituents are symmetrical with respect to each other.

8. The complex of claim 4 wherein the 4 and 7 substituents are asymmetrical with respect to each other.

9. The complex of claim 4 wherein the 4 and 7 substituents are symmetrical with respect to each other.

10. A method of treating a patient having cancerous cells affecting tissue comprising providing a cis-LPtCl₂ complex wherein L is a 4,4′-substituted-2,2′-bipyridine or a 4,7-substituted-1,10-phenanthroline compound comprising 2,2′-bipyridines and 1,10-phenanthrolines having alkyl groups appended in both the 4,4′-positions of the bipyridine and both of the 4,7-positions of the phenanthroline to the affected tissue.

11. The method of claim 10 wherein the alkyl group is selected from the group consisting of normal, branched and cyclic alkyl groups, alkyl groups with ether linkages, highly fluorinated alkyl group, highly fluorinated alkyl groups with ether linkages, hydroxyl terminated alkyl groups, hydroxyl-terminated alkyl groups with ether linkages and perfluorinated alkyl groups.

12. The method of claim 11 represented by a formula selected from the group consisting of (CH₂)ₙ(CH₃); (CH₂)ₙ—O—CH₃; CH₂(CH₂)ₙ(CF₂)ₓ₋₁CF₃(CClF)ₙ(CF₂)ₓ₋₁CF₃; and (CH₂)ₙ(CH₂OH) wherein n=0-5, m=0-3; and x=1-6]

13. The method of claim 10 wherein providing the complex to the affected tissue is performed by at least one of general administration of the complex and affected tissue targeted administration of the complex.

14. The method of claim 11 wherein L comprises a 4,4′-substituted 2,2′-bipyridine.

15. The method of claim 11 wherein L comprises a 4,7-substituted-1,10-phenanthroline.

16. The method of claim 12 wherein the 4 and 4′ substituents are asymmetrical with respect to each other.

17. The method of claim 12 wherein the 4 and 4′ substituents are symmetrical with respect to each other.

18. A method of treating a patient having cancerous cells affecting tissue comprising
providing a cancer cell growth reducing effective amount to the tissue comprising cancerous cells of a complex having a formula L-PtCl2 wherein L is a ligand selected from the group consisting of: 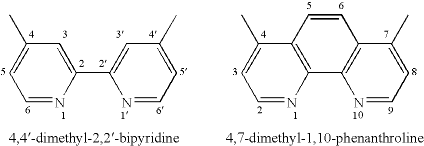
wherein the 4- and 4′-positions of the bipyridine and the 4 and 7-positions of the phenanthroline are independently substituted with alkyl groups.

19. The method of claim 18 wherein the alkyl groups are selected from the group consisting of normal, branched and cyclic alkyl groups, alkyl groups with ether linkages, highly fluorinated alkyl group, highly fluorinated alkyl groups with ether linkages, hydroxyl terminated alkyl groups, hydroxyl-terminate alkyl groups with ether linkages and perfluorinated alkyl groups.
